(19) United States
(12) Patent Application Publication
(10) Pub. No.: US 2026/0063994 A1
(43) Pub. Date: Mar. 5, 2026

(54) RADIATION-SENSITIVE COMPOSITION, METHOD FOR FORMING PATTERN AND ONIUM SALT COMPOUND

(71) Applicant: JSR CORPORATION, Tokyo (JP)

(72) Inventors: Hajime INAMI, Tokyo (JP); Fuyuki EGAWA, Tokyo (JP); Ryuichi NEMOTO, Tokyo (JP); Masayuki MIYAKE, Tokyo (JP); Kensuke MIYAO, Tokyo (JP)

(73) Assignee: JSR CORPORATION, Tokyo (JP)

(21) Appl. No.: 19/377,148

(22) Filed: Nov. 3, 2025

Related U.S. Application Data
(63) Continuation-in-part of application No. PCT/JP2024/016060, filed on Apr. 24, 2024.

(30) Foreign Application Priority Data
May 22, 2023 (JP) 2023-083930

(51) Int. Cl. G03F 7/004; C07C 25/18; C07C 309/25; C07C 309/27; C07C 309/42; C07C 309/43; C07C 309/44; C07C 309/51; C07C 309/58; C07C 309/59; C07C 323/66; C07C 381/12; C07D 307/64; C07D 307/68; C07D 327/06; C07D 327/08; C07D 333/34; C07D 333/40; C07D 333/46; C07D 333/76; C07D 335/02; C07D 335/12; C08F 20/18; C08F 20/22; C08F 20/28; C08F 20/38; G03F 7/00; G03F 7/038; G03F 7/039

(52) U.S. Cl. CPC G03F 7/0045; C07C 25/18; C07C 309/25; C07C 309/27; C07C 309/42; C07C 309/43; C07C 309/44; C07C 309/51; C07C 309/58; C07C 309/59; C07C 323/66; C07C 381/12; C07D 307/64; C07D 307/68; C07D 327/06; C07D 327/08; C07D 333/34; C07D 333/40; C07D 333/46; C07D 333/76; C07D 335/02; C07D 335/12; C08F 20/18; C08F 20/22; C08F 20/28; C08F 20/38; G03F 7/0382; G03F 7/0388; G03F 7/0397; C07C 2601/08; C07C 2601/14; C07C 2601/16; C07C 2602/20; C07C 2603/74; C07C 2603/86; G03F 7/70033

(57) ABSTRACT

A radiation-sensitive composition includes: an onium salt compound represented by formula (1); a polymer including a structural unit (I) which includes an acid-dissociable group; and a solvent. W is a cyclic structure having 3 to 40 ring members formed together with the two carbon atoms; a formula between the two carbon atoms below represents a single bond or a double bond, [symbol] , A is one of groups represented by formulae (A-1) to (A-7); R¹ is a monovalent organic group having 1 to 20 carbon atoms, a cyano group, a nitro group, a carboxy group, a hydroxy group, an amino group, a halogen atom, or a thiol group, when there are a plurality of R¹s, the plurality of R¹s are same as or different from each other; m₁ is an integer of 0 to 4; and Z⁺ is a monovalent radiation-sensitive onium cation.

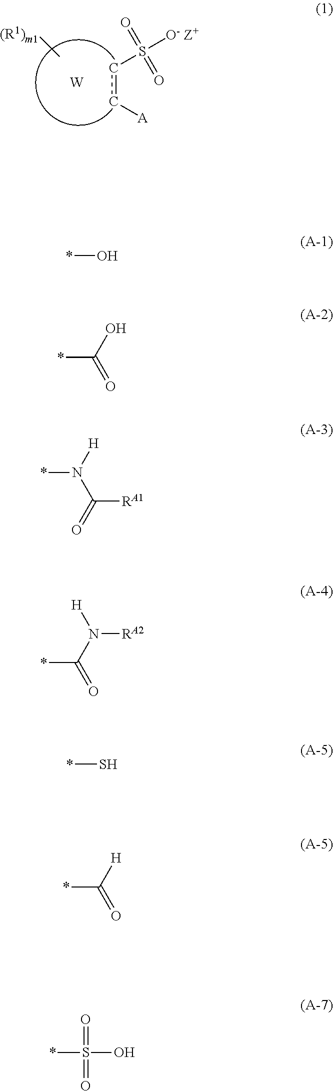

### CROSS-REFERENCE TO RELATED APPLICATIONS

The present application is a continuation-in-part application of International Patent Application No. PCT/JP2024/016060 filed Apr. 24, 2024, which claims priority to Japanese Patent Application No. 2023-083930 filed May 22, 2023. The contents of these applications are incorporated herein by reference in their entirety.

### BACKGROUND OF THE DISCLOSURE

### Technical Field

The present disclosure relates to a radiation-sensitive composition, a method for forming a pattern and an onium salt compound.

### Background Art

A photolithography technology using a resist composition has been used for the fine circuit formation in a semiconductor device. As the representative procedure, for example, a resist pattern is formed on a substrate by generating an acid by irradiating the coating of the resist composition with a radioactive ray through a mask pattern, and then reacting in the presence of the acid as a catalyst to generate the difference of solubility of a polymer into an alkaline or organic developer between an exposed part and a non-exposed part.

In the photolithography technique, the micronization of the pattern is promoted by using a short-wavelength radioactive ray such as an ArF excimer laser or by using an immersion exposure method (liquid immersion lithography) in which exposure is performed in a state in which a space between a lens of an exposure apparatus and a resist film is filled with a liquid medium. As a next-generation technology, lithography using shorter wavelength radiation such as electron beams, X-rays and EUV (extreme ultraviolet rays) is also being considered.

As for the photoacid generator, which is a main component of the resist composition, perfluoroalkylsulfonic acid capable of imparting strong acidity is often used from the viewpoint of improving sensitivity, resolution, etc. On the other hand, with the recent increase in environmental awareness, a photoacid generator having a reduced fluorine atom content has been studied (see JP-B-7015295).

### SUMMARY

According to an aspect of the present disclosure, a radiation-sensitive composition includes: an onium salt compound represented by formula (1); a polymer including a structural unit (I) which includes an acid-dissociable group; and a solvent.

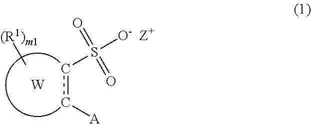

W is a cyclic structure having 3 to 40 ring members formed together with the two carbon atoms; a formula between the two carbon atoms below represents a single bond or a double bond, [symbol] ; A is a group represented by formula (A-1), a group represented by formula (A-2), a group represented by formula (A-3), a group represented by formula (A-4), a group represented by formula (A-5), a group represented by formula (A-6), or a group represented by formula (A-7).

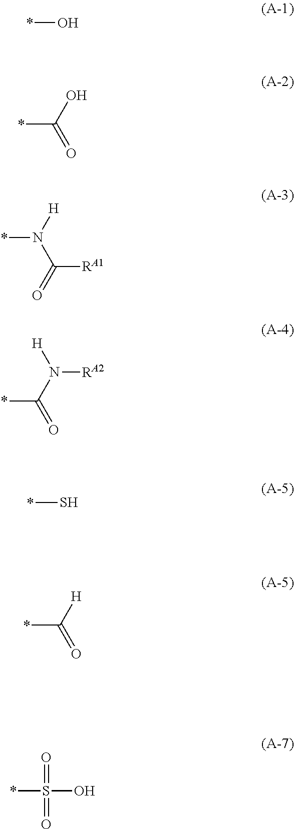

In the formulas (A-3) and (A-4), R^{A1} and R^{A2} are each independently a monovalent hydrocarbon group having 1 to 20 carbon atoms, and * is a bond with a carbon atom. R¹ is a monovalent organic group having 1 to 20 carbon atoms, a cyano group, a nitro group, a carboxy group, a hydroxy group, an amino group, a halogen atom, or a thiol group, when there are a plurality of R¹s, the plurality of R¹s are same as or different from each other; m₁ is an integer of 0 to 4; and Z⁺ is a monovalent radiation-sensitive onium cation.

According to another aspect of the present disclosure, a method for forming a pattern, includes: directly or indirectly applying the above-described radiation-sensitive composition to a substrate to form a resist film; exposing the resist film to light; and developing the exposed resist film with a developer.

According to a further aspect of the present disclosure, an onium salt compound is represented by formula (1).

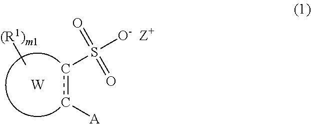

W is a cyclic structure having 3 to 40 ring members formed together with the two carbon atoms; a formula between the two carbon atoms below represents a single bond or a double bond, [symbol] ; A is a group represented by formula (A-1), a group represented by formula (A-2), a group represented by formula (A-3), a group represented by formula (A-4), a group represented by formula (A-5), a group represented by formula (A-6), or a group represented by formula (A-7).

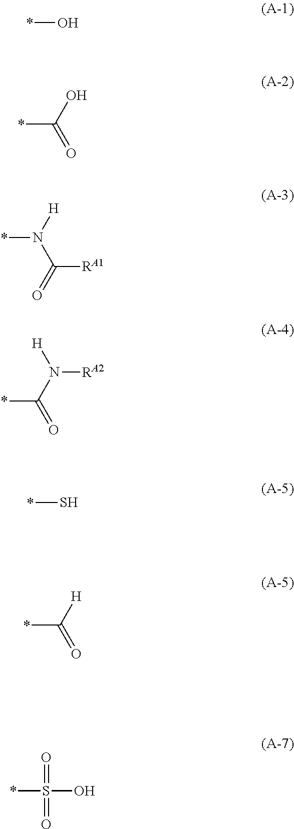

In the formulas (A-3) and (A-4), R^{A1} and R^{A2} are each independently a monovalent hydrocarbon group having 1 to 20 carbon atoms, and * is a bond with a carbon atom. R¹ is a monovalent organic group having 1 to 20 carbon atoms, a cyano group, a nitro group, a carboxy group, a hydroxy group, an amino group, a halogen atom, or a thiol group, when there are a plurality of R¹s, the plurality of R¹s are same as or different from each other; m₁ is an integer of 0 to 4; and Z⁺ is a monovalent radiation-sensitive onium cation.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed.

Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

Even in the case of a photoacid generator having a reduced fluorine atom content, various resist performances equivalent to or higher than conventional ones are required in terms of sensitivity, line width, LWR (Line Width Roughness) performance indicating variations in line width of a resist pattern, CDU (Critical Dimension Uniformity) performance, and the like.

On the other hand, as a result of studies of a photoacid generator having a reduced fluorine atom content, the present inventors have found that the storage stability of the resist composition may be deteriorated.

Since the radiation-sensitive composition of the present disclosure contains the onium salt compound (1) as a radiation-sensitive acid generator, the radiation-sensitive composition can exhibit excellent sensitivity, LWR performance, and CDU performance in pattern formation, and also has good storage stability. Although not bound by any theory, the reason for this can be presumed as follows.

In the onium salt compound (1), a specific polar group containing a hydrogen atom is bonded to the carbon atom adjacent to the carbon atom to which the sulfonate anion is bonded. Due to this steric configuration, a hydrogen bond is generated between the sulfonate anion and the hydrogen atom of the specific polar group, the anion is stabilized, and as a result, the strength of a generated acid is so increased that the various resist performances are exhibited. On the other hand, in a conventional photoacid generator in which an electron withdrawing group (including an ester bond) is disposed around the sulfonate anion, the sensitivity may be extremely lowered depending on the positional relationship between the electron withdrawing group and the sulfonate anion, the degree of freedom of the sulfonate anion, and the like, or the storage stability may be deteriorated due to decomposition of the ester bond or the like. The onium salt compound (1) adopts strong acidification in which anion stabilization by hydrogen bonding between the sulfonate anion and the specific polar group each bonded to the β-position carbon in the cyclic structure (ortho position when the cyclic structure is an aromatic ring) is used as the mechanism of action, rather than strong acidification by arrangement of the electron withdrawing group. This makes it possible to reduce the influence of the electron withdrawing group and the degree of freedom of the sulfonate anion, so that the sensitivity can be controlled within an appropriate range. Furthermore, it is presumed that the destabilization (decomposition or the like) of the compound itself caused by the electron withdrawing group is suppressed, so that good storage stability can be exhibited. The organic group refers to a group containing at least one carbon atom.

In the method for forming a pattern of the present disclosure, since the radiation-sensitive composition having excellent sensitivity, LWR performance, and CDU performance in pattern formation and having good storage stability is used, a high-quality resist pattern can be formed with good yield.

Since the onium salt compound of the present disclosure has the above specific structure, when the onium salt compound is applied to a radiation-sensitive composition, the onium salt compound has good storage stability, and can exhibit excellent sensitivity, LWR performance, and CDU performance in pattern formation.

Hereinbelow, embodiments of the present disclosure will be described in detail, but the present disclosure is not limited to these embodiments. Combinations of suitable embodiments are also preferable.

### <Radiation-Sensitive Composition>

The radiation-sensitive composition (hereinafter, also simply referred to as “composition”) according to the present embodiment includes the onium salt compound (1), a polymer, and a solvent. The radiation-sensitive composition further includes an acid diffusion controlling agent, as necessary. The composition may contain another optional component as long as the effects of the present disclosure are not impaired.

### (Onium Salt Compound (1))

The onium salt compound (1) is represented by the formula (1), and functions as a radiation-sensitive acid generator that generates an acid in response to irradiation with radiation.

The cyclic structure having 3 to 40 ring members formed together with two carbon atoms represented by W is not particularly limited, but an alicyclic hydrocarbon structure having 3 to 20 carbon atoms, an aromatic hydrocarbon structure having 6 to 20 carbon atoms, an aliphatic heterocyclic structure having 3 to 20 carbon atoms, or an aromatic heterocyclic structure having 5 to 20 carbon atoms is preferable. Alternatively, these ring structures may be combined. Examples of an aspect of the combination include a fused ring in which two adjacent rings share one side (two adjacent interatomic bonds), a ring assembly in which two adjacent rings are bonded by a single bond, and a spiro ring in which two adjacent rings share one carbon atom. As an aspect of the combination, a fused ring is preferable.

As the alicyclic hydrocarbon structure having 3 to 20 carbon atoms, a structure corresponding to a monovalent alicyclic hydrocarbon group having 3 to 20 carbon atoms can be suitably adopted. Examples of the monovalent alicyclic hydrocarbon group having 3 to 20 carbon atoms include monovalent monocyclic or polycyclic saturated hydrocarbon groups and monocyclic or polycyclic unsaturated hydrocarbon groups. As the monocyclic saturated hydrocarbon groups, a cyclopentyl group, a cyclohexyl group, a cycloheptyl group, and a cyclooctyl group are preferable. As the polycyclic saturated hydrocarbon groups, bridged alicyclic hydrocarbon groups such as a norbornyl group, an adamantyl group, a tricyclodecyl group, and a tetracyclododecyl group are preferable. Examples of the monocyclic unsaturated hydrocarbon group include monocyclic cycloalkenyl groups such as a cyclopropenyl group, a cyclobutenyl group, a cyclopentenyl group, and a cyclohexenyl group. Examples of the polycyclic unsaturated hydrocarbon group include polycyclic cycloalkenyl groups such as a norbornenyl group, a tricyclodecenyl group, and a tetracyclododecenyl group. The bridged alicyclic hydrocarbon group refers to a polycyclic alicyclic hydrocarbon group in which two carbon atoms that constitute an alicyclic ring and are not adjacent to each other are bonded by a linking group containing one or more carbon atoms.

As the aromatic hydrocarbon structure having 6 to 20 carbon atoms, a structure corresponding to a monovalent aromatic hydrocarbon group having 6 to 20 carbon atoms can be suitably adopted. Examples of the monovalent aromatic hydrocarbon group having 6 to 20 carbon atoms include: aryl groups such as a phenyl group, a tolyl group, a xylyl group, a naphthyl group, and an anthryl group; and aralkyl groups such as a benzyl group, a phenethyl group, and a naphthylmethyl group.

Examples of the aliphatic heterocyclic structure include:
oxygen atom-containing aliphatic heterocyclic structures such as oxirane, tetrahydrofuran, tetrahydropyran, dioxolane, and dioxane;
nitrogen atom-containing aliphatic heterocyclic structures such as aziridine, pyrrolidine, piperidine, and piperazine;
sulfur atom-containing aliphatic heterocyclic structures such as thietane, thiolane, and thiane; and
aliphatic heterocyclic structures containing a plurality of heteroatoms, such as morpholine, 1,2-oxathiolane, and 1,3-oxathiolane.

Examples of the aliphatic heterocyclic structure include a lactone structure, a cyclic carbonate structure, a sultone structure, and a structure containing a cyclic acetal and a cyclic ketone. Examples of such structures include structures represented by the following formulas (H-1) to (H-12). Although all carbon-carbon bonds of the following structural formulas are saturated bonds, an unsaturated bond may be introduced as long as the valence is permitted.

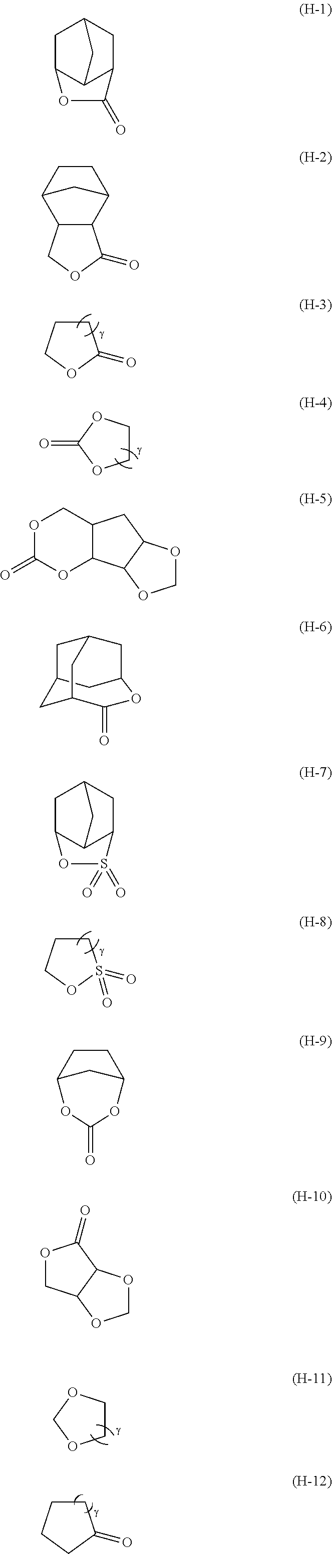

In the above formula, γ is an integer of 1 to 3.

Examples of the aromatic heterocyclic structure having 5 to 20 carbon atoms include:
oxygen atom-containing aromatic heterocyclic structures such as furan, pyran, benzofuran, and benzopyran;
nitrogen atom-containing aromatic heterocyclic structures such as pyrrole, imidazole, pyridine, pyrimidine, pyrazine, indole, quinoline, isoquinoline, acridine, phenazine, and carbazole;
sulfur atom-containing aromatic heterocyclic structures such as thiophene; and
aromatic heterocyclic structures containing a plurality of hetero atoms such as thiazole, benzothiazole, thiazine, and oxazine.

When the following formula between carbon atoms in W in the formula (1) represents a double bond, this double bond also includes a bond of a conjugated system when the cyclic structure exhibits aromaticity.

[symbol]

Among them, as the cyclic structure having 3 to 40 ring members of W, a polycyclic alicyclic hydrocarbon structure having 6 to 14 carbon atoms, an aromatic hydrocarbon structure having 6 to 12 carbon atoms, a monocyclic aliphatic unsaturated heterocyclic structure having 5 to 8 carbon atoms, or an aromatic heterocyclic structure having 5 to 8 carbon atoms is more preferable.

Examples of the monovalent hydrocarbon group having 1 to 20 carbon atoms represented by R^{A1} and R^{A2} in the formulas (A-3) and (A-4) include a monovalent chain hydrocarbon group having 1 to 20 carbon atoms, a monovalent alicyclic hydrocarbon group having 3 to 20 carbon atoms, a monovalent aromatic hydrocarbon group having 6 to 20 carbon atoms, or combinations thereof.

Examples of the monovalent chain hydrocarbon group having 1 to 20 carbon atoms may include a linear or branched saturated chain hydrocarbon group having 1 to 20 carbon atoms or a linear or branched unsaturated chain hydrocarbon group having 1 to 20 carbon atoms.

As the monovalent alicyclic hydrocarbon group having 3 to 20 carbon atoms, the monovalent alicyclic hydrocarbon group having 3 to 20 carbon atoms shown in W can be suitably employed.

As the monovalent aromatic hydrocarbon group having 6 to 20 carbon atoms, the monovalent aromatic hydrocarbon group having 6 to 20 carbon atoms shown in W can be suitably employed.

As the monovalent hydrocarbon group having 1 to 20 carbon atoms represented by R^{A1} and R^{A2} in the formulas (A-3) and (A-4), a monovalent chain hydrocarbon group having 1 to 20 carbon atoms is preferable, a monovalent chain saturated hydrocarbon group having 1 to 10 carbon atoms is more preferable, and a monovalent branched chain saturated hydrocarbon group having 1 to 5 carbon atoms is still more preferable.

Examples of the monovalent organic group having 1 to 20 carbon atoms represented by R¹ in the formula (1) include a monovalent hydrocarbon group having 1 to 20 carbon atoms, a group (hereinafter, also referred to as a “group (α)”) in which some or all of hydrogen atoms contained in the hydrocarbon group are substituted with a substituent, a group (hereinafter, also referred to as a “group (β)”) containing —CO—, —CS—, —O—, —S—, —SO₂—, —NR′—, or a combination of two or more thereof between carbon atoms of the hydrocarbon group or the group (a), or a combination thereof. R′ is a hydrogen atom or a monovalent hydrocarbon group having 1 to 10 carbon atoms.

As the monovalent hydrocarbon group having 1 to 20 carbon atoms, a monovalent hydrocarbon group having 1 to 20 carbon atoms represented by R^{A1} and R^{A2} in the formulas (A-3) and (A-4) can be suitably employed.

Examples of the substituent that substitutes some or all of the hydrogen atoms of the organic group include a halogen atom such as a fluorine atom, a chlorine atom, a bromine atom, or an iodine atom; a hydroxy group; a carboxy group; a cyano group; a nitro group; an alkyl group; an alkoxy group; an alkoxycarbonyl group; an alkoxycarbonyloxy group; an acyl group; an acyloxy group, or groups obtained by substituting a hydrogen atom of these groups with a halogen atom; and an oxo group (═O).

Examples of the halogen atom represented by R¹ in the formula (1) include a fluorine atom, a chlorine atom, a bromine atom, and an iodine atom. As the halogen atom, a chlorine atom, a bromine atom, and an iodine atom are preferable, and an iodine atom is more preferable.

m1 is preferably an integer of 0 to 3, more preferably an integer of 0 to 2.

Specific examples of the anion of the onium salt compound (1) include, but are not limited to, structures represented by the following formulas (1-1-1) to (1-1-22).

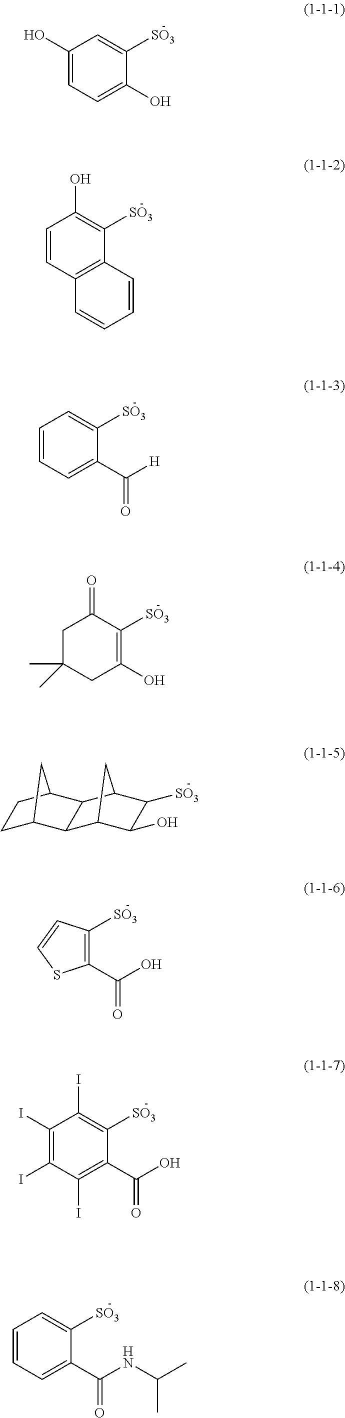

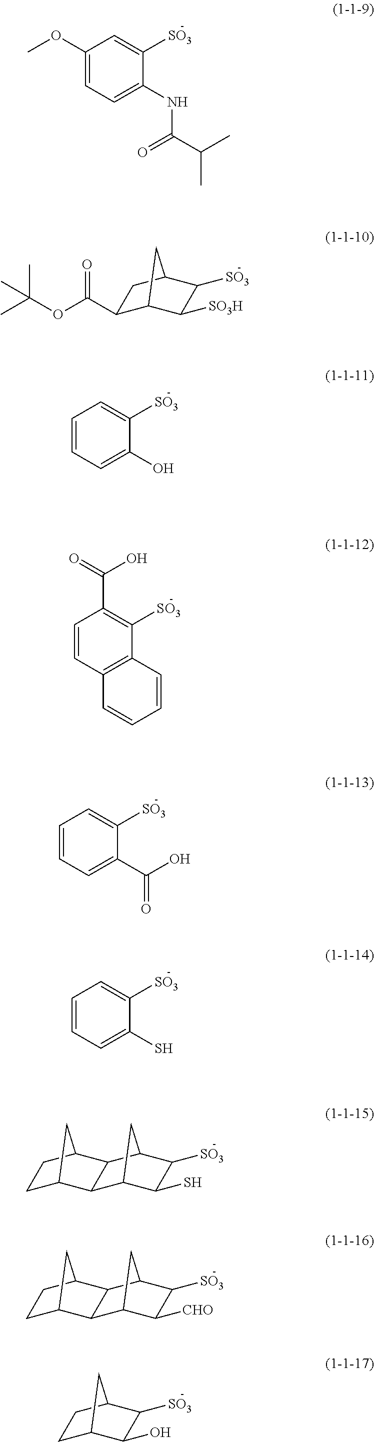

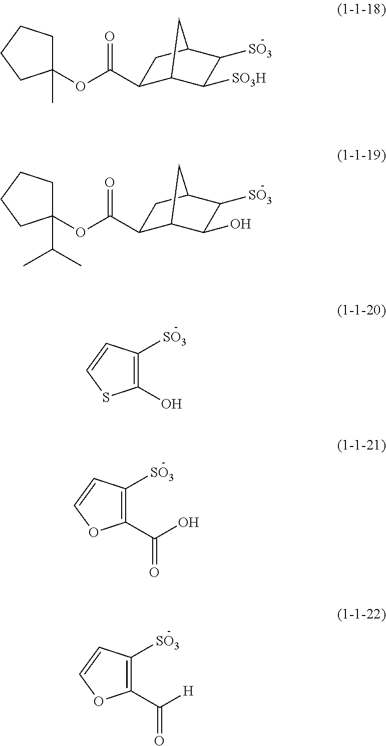

An example of the monovalent radiation-sensitive onium cation represented by Z⁺ is a radioactive ray-degradable onium cation containing an element such as S, I, O, N, P, Cl, Br, F, As, Se, Sn, Sb, Te, or Bi. Examples of such a radioactive ray-degradable onium cation include a sulfonium cation, a tetrahydrothiophenium cation, a iodonium cation, a phosphonium cation, a diazonium cation, and a pyridinium cation. Among them, a sulfonium cation or a iodonium cation is preferred. Z⁺ preferably contains at least one aromatic ring, and more preferably is a monovalent sulfonium cation containing at least one aromatic ring or a monovalent iodonium cation containing at least one aromatic ring. The sulfonium cation or the iodonium cation is preferably represented by any of the formulas (X-1) to (X-6).

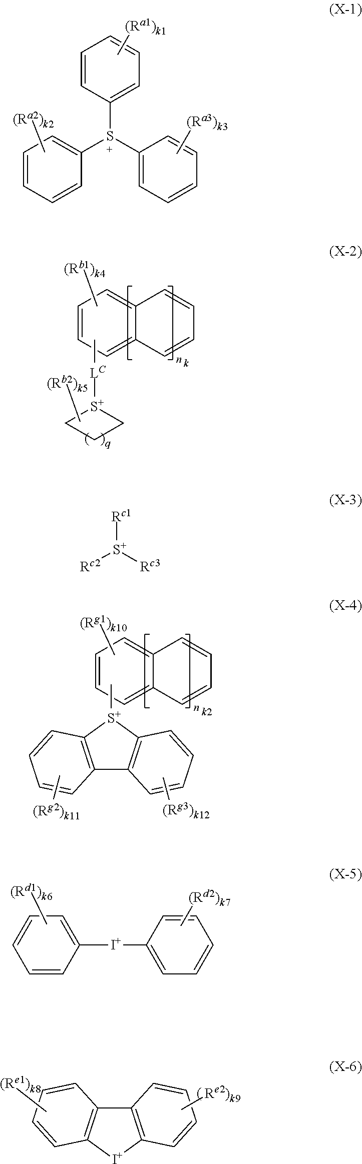

In the formula (X-1), R^{a1}, R^{a2} and R^{a3} are each independently a substituted or unsubstituted, straight or branched chain alkyl group, alkoxy group, or alkoxycarbonyloxy group having a carbon number of 1 to 12; a substituted or unsubstituted, monocyclic or polycyclic cycloalkyl group having a carbon number of 3 to 12; a substituted or unsubstituted aromatic hydrocarbon group having a carbon number of 6 to 12; a hydroxy group, a halogen atom, —OSO₂—R^{P}, —SO₂—R^{Q} or —S—R^{T}; or a ring structure obtained by combining two or more of these groups. The ring structure may contain heteroatoms such as O and S between the carbon-carbon bonds forming the skeleton. R^{P}, R^{Q} and R^{T} are each independently a substituted or unsubstituted, straight or branched chain alkyl group having a carbon number of 1 to 12; a substituted or unsubstituted alicyclic hydrocarbon group having a carbon number of 5 to 25; and a substituted or unsubstituted aromatic hydrocarbon group having a carbon number of 6 to 12. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the formula (X-2), R^{b1} is a substituted or unsubstituted, straight chain or branched alkyl group or alkoxy group having a carbon number of 1 to 20; a substituted or unsubstituted acyl group having a carbon number of 2 to 8; or a substituted or unsubstituted aromatic hydrocarbon group having a carbon number of 6 to 8; or a hydroxy group. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, straight chain or branched alkyl group having a carbon number of 1 to 7; or a substituted or unsubstituted aromatic hydrocarbon group having a carbon number of 6 or 7. L^{C} is a single bond or divalent linking group. k5 is an integer of 0 to 4. When there are a plurality of R^{b2}, a plurality of R^{b}2 may be each identical or different. A plurality of R^{b2} may represent a ring structure obtained by combining them. q is an integer of 0 to 3. In the formula, the ring structure containing S⁺ may contain a heteroatom such as O or S between the carbon-carbon bonds forming the skeleton.

In the formula (X-3), R^{c1}, R^{c2} and R^{c3} are each independently a substituted or unsubstituted, straight or branched chain alkyl group having a carbon number of 1 to 12, or a substituted or unsubstituted aryl group having 6 to 12 carbon atoms. At least one selected from the group consisting of R^{c1}, R^{c2} and R^{c3} is a substituted or unsubstituted linear or branched alkyl group having 1 to 12 carbon atoms.

In the formula (X-4), R^{g1} is a substituted or unsubstituted linear or branched alkyl or alkoxy group having 1 to 20 carbon atoms, a substituted or unsubstituted acyl group having 2 to 8 carbon atoms, a substituted or unsubstituted aromatic hydrocarbon group having 6 to 8 carbon atoms, or a hydroxy group. nₖ₂ is 0 or 1. When nₖ₂ is 0, k10 is an integer of 0 to 4, and when nₖ₂ is 1, k10 is an integer of 0 to 7. When there are two or more R^{g1}s, the two or more R^{g1}s are the same or different from each other, and may represent a cyclic structure formed by combining them together. R^{g2} and R^{g3} are each independently a substituted or unsubstituted linear or branched alkyl, alkoxy, or alkoxycarbonyloxy group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxyl group, a halogen atom, or a ring structure formed by combining two or more of these groups together. K11 and k12 are each independently an integer of 0 to 4. When there are two or more R^{g2}s and two or more R^{g3}s, the two or more R^{g2}s may be the same or different from each other, and the two or more R^{g3}s may be the same or different from each other.

In the formula (X-5), R^{d1} and R^{d2} are each independently a substituted or unsubstituted, straight or branched chain alkyl group, alkoxy group or alkoxycarbonyl group having a carbon number of 1 to 12; a substituted or unsubstituted aromatic hydrocarbon group having a carbon number of 6 to 12; a halogen atom; a halogenated alkyl group having a carbon number of 1 to 4; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the formula (X-6), R^{e1} and R^{e2} are each independently a halogen atom; a substituted or unsubstituted straight or branched chain alkyl group having a carbon number of 1 to 12; or a substituted or unsubstituted aromatic hydrocarbon group having a carbon number of 6 to 12. k8 and k9 are each independently an integer of 0 to 4.

Specific examples of the radiation-sensitive onium cation include, but not limited thereto, the structures represented by the formulas (1-2-1) to (1-2-50).

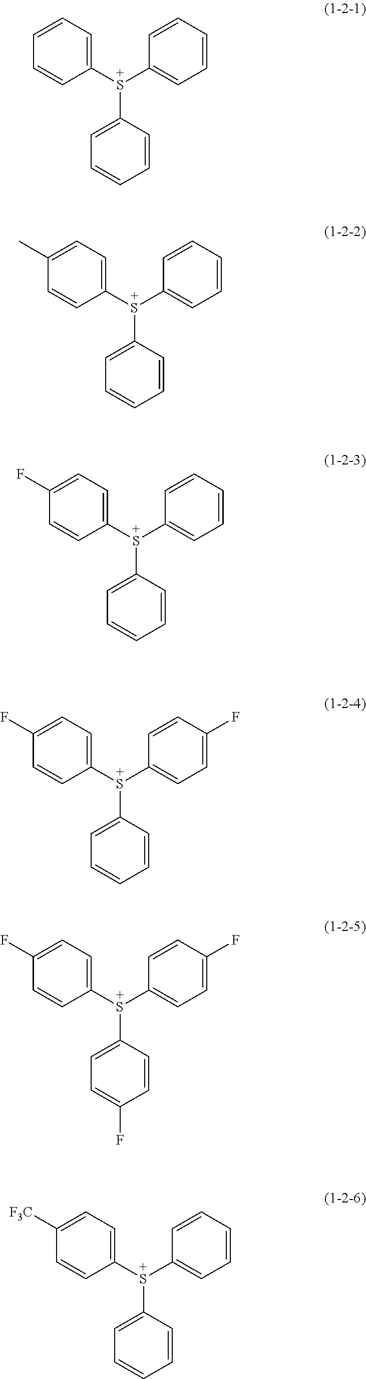

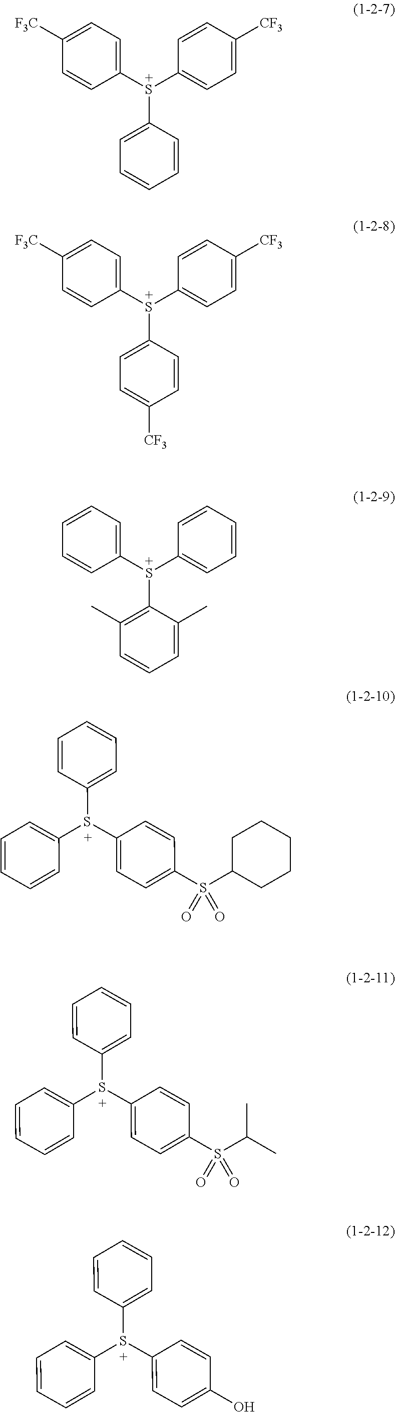

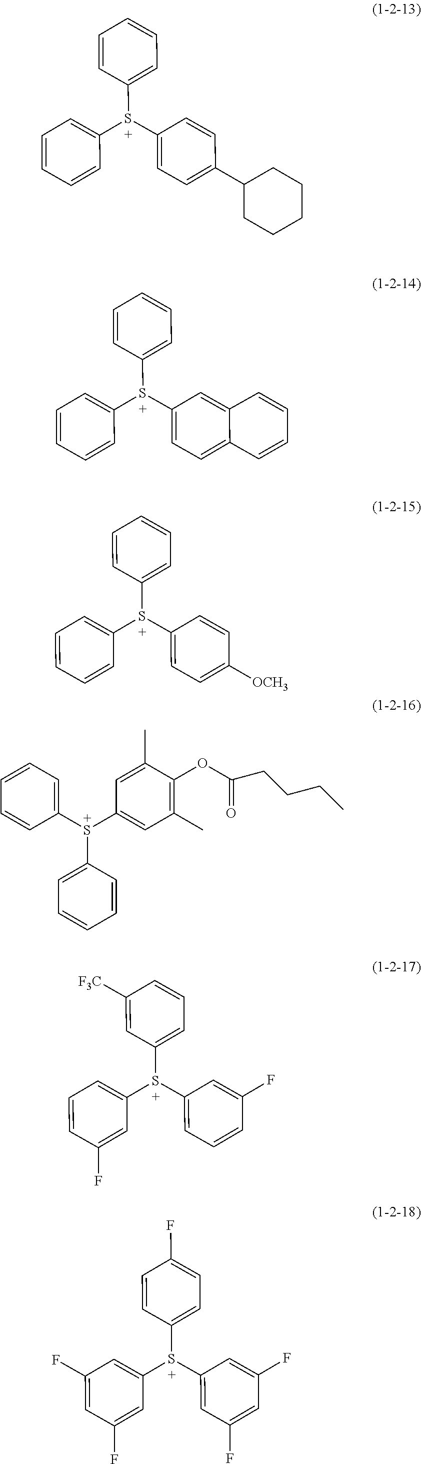

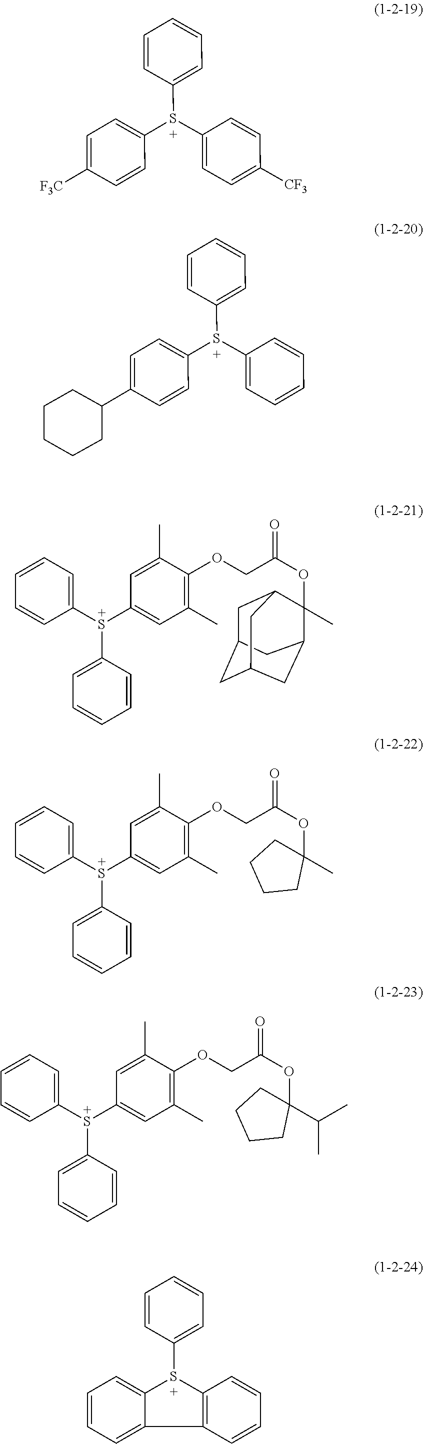

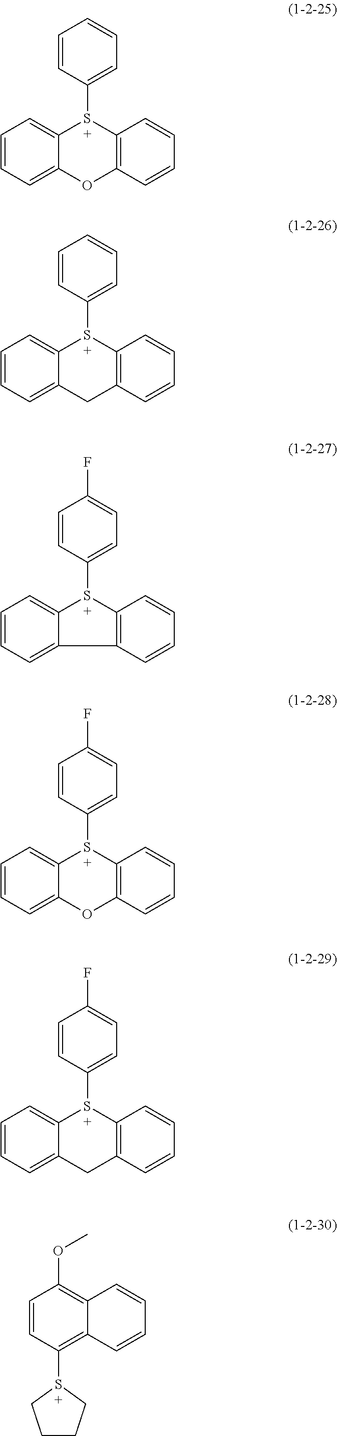

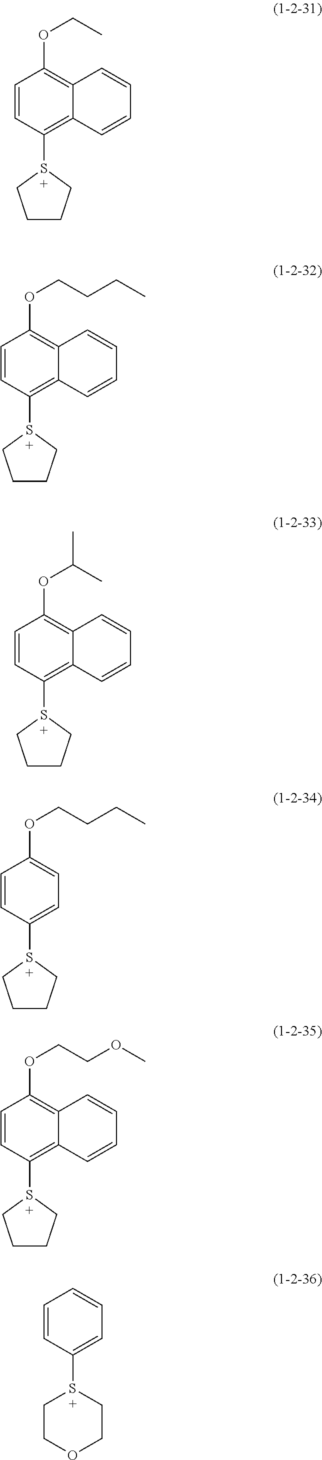

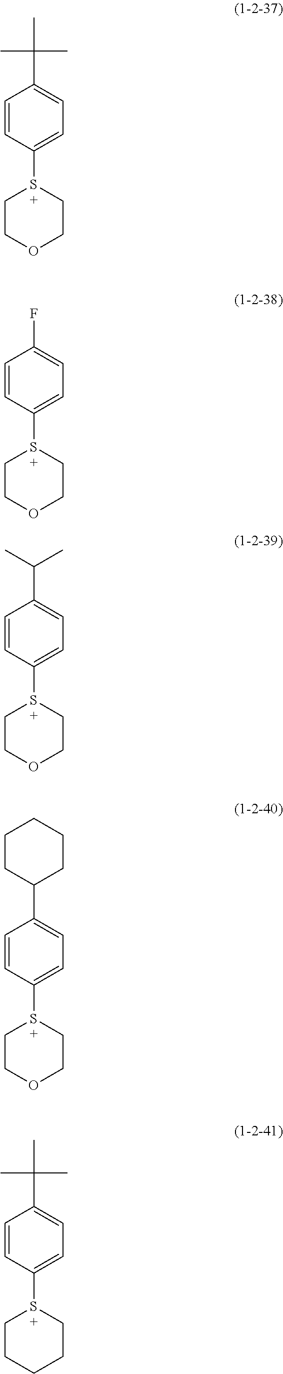

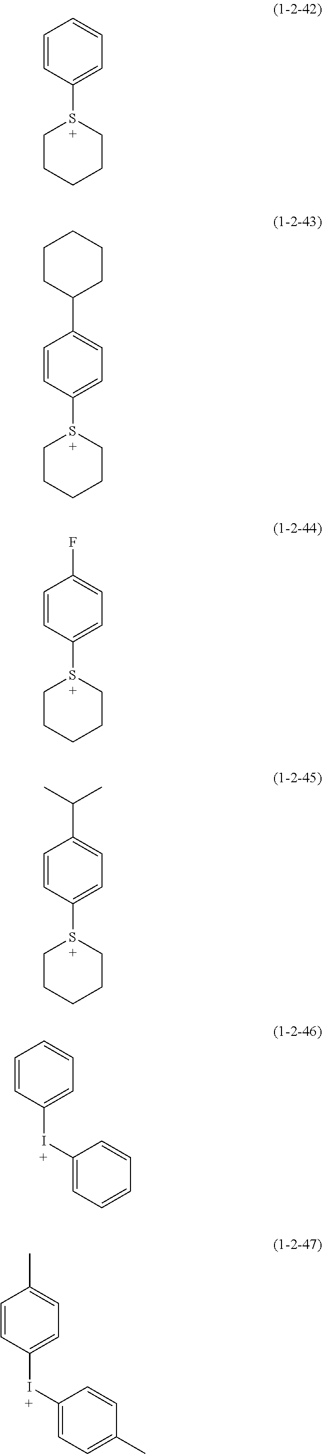

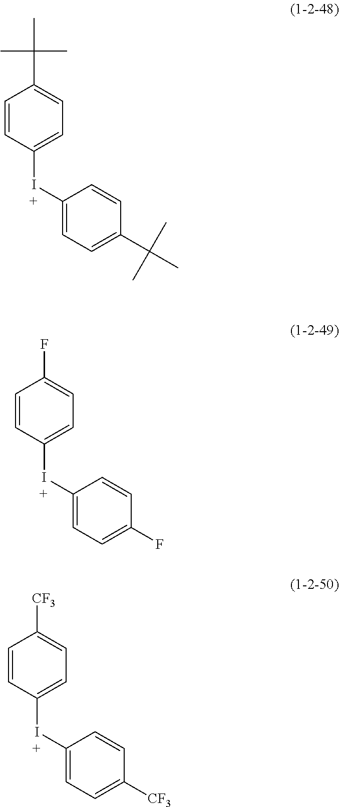

The onium salt compound (1) is obtained by appropriately combining the aforementioned anion and the aforementioned radiation-sensitive onium cations. Specific examples thereof include, but are not particularly limited to, structures represented by formulae (1-1) to (1-22).

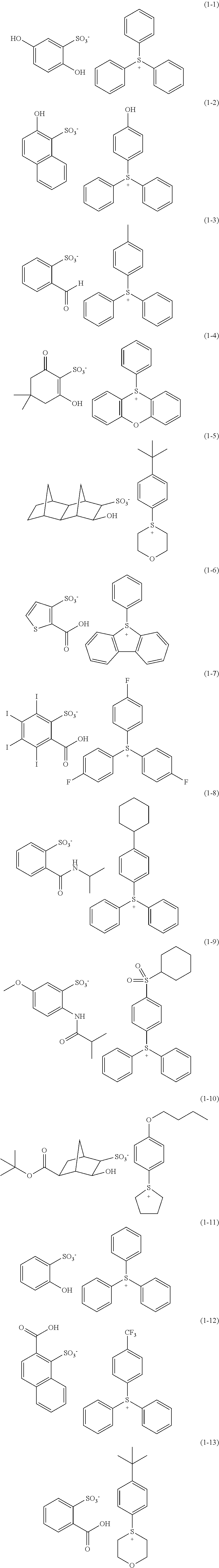

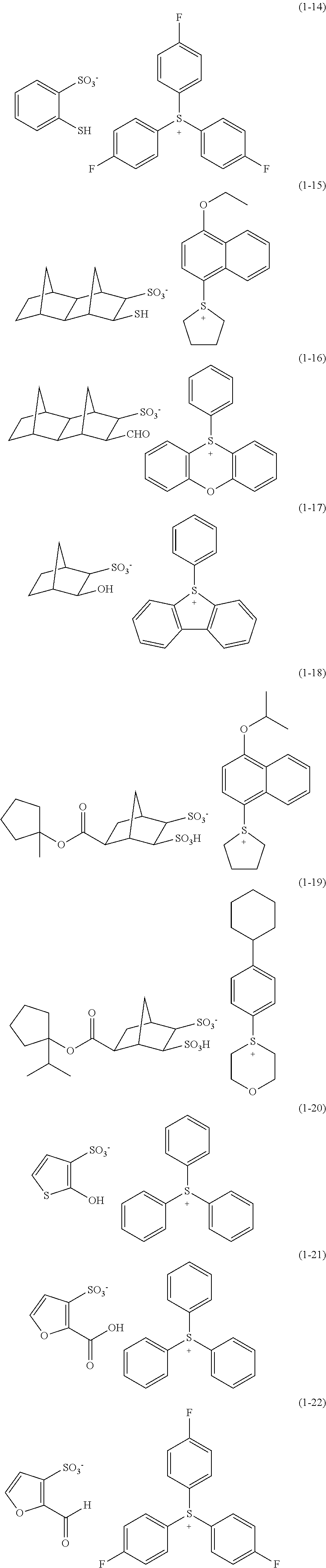

The lower limit of the content of the onium salt compound (1) (when plural kinds of onium salt compounds (1) are contained, the total content thereof) is preferably 1 part by mass, more preferably 3 parts by mass, still more preferably 5 parts by mass, particularly preferably 8 parts by mass with respect to 100 parts by mass of the polymer described later. The upper limit of the content is preferably 40 parts by mass, more preferably 30 parts by mass, still more preferably 20 parts by mass, even more preferably 15 parts by mass. The content of the onium salt compound (1) is appropriately selected depending on the type of a polymer to be used, exposure conditions, required sensitivity, and the like. This makes it possible to exhibit excellent sensitivity, LWR performance, and CDU performance in resist pattern formation while maintaining the storage stability of the composition.

### (Method for Synthesizing Onium Salt Compound (1))

Regarding the method for synthesizing the onium salt compound (1), a case where A in the formula (1) is a hydroxy group will be described. The target onium salt compound (1) can be synthesized by reacting a hydroxysulfonate salt product (for example, a potassium salt or the like) corresponding to the target anion structure with an onium cation halide salt (for example, a bromide salt or the like) corresponding to the onium cation to promote salt exchange. Similarly, the onium salt compound (1) having another structure can be synthesized by appropriately selecting starting materials and precursors corresponding to the anion and the onium cation.

### (Polymer)

The polymer is an aggregate of polymers having a structural unit (hereinafter, also referred to as “structural unit (I)”) containing an acid-dissociable group (hereinafter, this polymer is also referred to as “base polymer”). The “acid-dissociable group” refers to a group that substitutes for a hydrogen atom of a carboxy group, a phenolic hydroxyl group, an alcoholic hydroxyl group, a sulfo group, or the like, and is dissociated by the action of an acid. The radiation-sensitive composition is excellent in pattern-forming performance because the polymer has the structural unit (I).

In addition to the structural unit (I), the base polymer preferably has a structural unit (II) containing at least one selected from the group consisting of a lactone structure, a cyclic carbonate structure, and a sultone structure described later, and may have another structural unit other than the structural units (I) and (II). Each of the structural units will be described below.

### [Structural Unit (I)]

The structural unit (I) contains an acid-dissociable group. The structural unit (I) is not particularly limited as long as it contains an acid-dissociable group. Examples of such a structural unit (I) include a structural unit having a tertiary alkyl ester moiety, a structural unit having a structure obtained by substituting the hydrogen atom of a phenolic hydroxyl group with a tertiary alkyl group, and a structural unit having an acetal bond. From the viewpoint of improving the pattern-forming performance of the radiation-sensitive composition, a structural unit represented by the formula (3) (hereinafter also referred to as a “structural unit (I-1)”) is preferred.

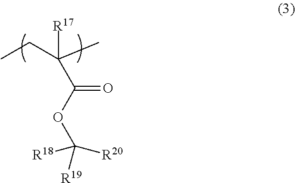

In the formula (3), R¹⁷ is a hydrogen atom, a fluorine atom, a methyl group, or a trifluoromethyl group, R¹⁸ is a monovalent hydrocarbon group having 1 to 20 carbon atoms, R¹⁹ and R²⁰ are each independently a monovalent chain hydrocarbon group having 1 to 10 carbon atoms or a monovalent alicyclic hydrocarbon group having 3 to 20 carbon atoms or R¹⁹ and R²⁰ taken together represent a divalent alicyclic group having 3 to 20 carbon atoms formed together with the carbon atom to which they are bonded.

As R¹⁷, from the viewpoint of the copolymerizability of a monomer that affords the structural unit (I-1), a hydrogen atom and a methyl group are preferable, and a methyl group is more preferable.

As the monovalent hydrocarbon group having 1 to 20 carbon atoms represented by R¹⁸, a monovalent hydrocarbon group having 1 to 20 carbon atoms represented by R^{A1} and R^{A2} in the formulas (A-3) and (A-4) can be suitably employed.

As R¹⁸, a linear or branched saturated hydrocarbon group having 1 to 10 carbon atoms, an alicyclic hydrocarbon group having 3 to 20 carbon atoms, and an aromatic hydrocarbon group having 6 to 10 carbon atoms are preferable.

The divalent alicyclic group having 3 to 20 carbon atoms formed by R¹⁹ and R²⁰ together with the carbon atom to which R¹⁹ and R²⁰ are bonded is not particularly limited as long as it is a group obtained by removing two hydrogen atoms from the same carbon atom constituting a carbon ring of a monocyclic or polycyclic alicyclic hydrocarbon having the above-described carbon number. The divalent alicyclic group having 3 to 20 carbon atoms may either be a monocyclic hydrocarbon group or a polycyclic hydrocarbon group. The polycyclic hydrocarbon group may either be a bridged alicyclic hydrocarbon group or a condensed alicyclic hydrocarbon group and may either be a saturated hydrocarbon group or an unsaturated hydrocarbon group. It is to be noted that the condensed alicyclic hydrocarbon group refers to a polycyclic alicyclic hydrocarbon group in which two or more alicyclic rings share their sides (bond between two adjacent carbon atoms).

When the monocyclic alicyclic hydrocarbon group is a saturated hydrocarbon group, preferred examples thereof include a cyclopentanediyl group, a cyclohexanediyl group, a cycloheptanediyl group, and a cyclooctanediyl group. When the monocyclic alicyclic hydrocarbon group is an unsaturated hydrocarbon group, preferred examples thereof include a cyclopentenediyl group, a cyclohexenediyl group, a cycloheptenediyl group, a cyclooctenediyl group, and a cyclodecenediyl group. The polycyclic alicyclic hydrocarbon group is preferably a bridged alicyclic saturated hydrocarbon group, and preferred examples thereof include a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-2,2-diyl group (adamantane-2,2-diyl group).

Among them, R¹⁸ is preferably an alkyl group having 1 to 4 carbon atoms or a phenyl group, and the alicyclic structure formed by R¹⁹ and R²⁰ together with the carbon atom to which they are bonded is preferably a polycyclic or monocyclic cycloalkane structure.

Examples of the structural unit (I-1) include structural units represented by the formulas (3-1) to (3-8) (hereinafter also referred to as “structural units (I-1-1) to (I-1-8)”).

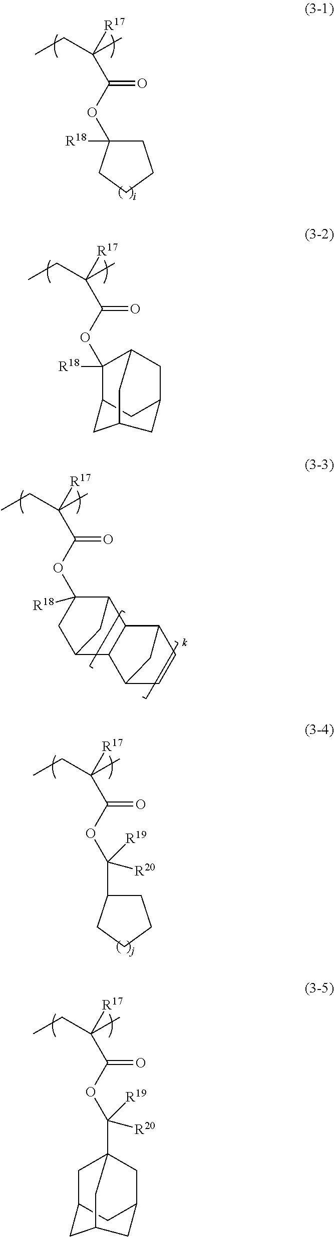

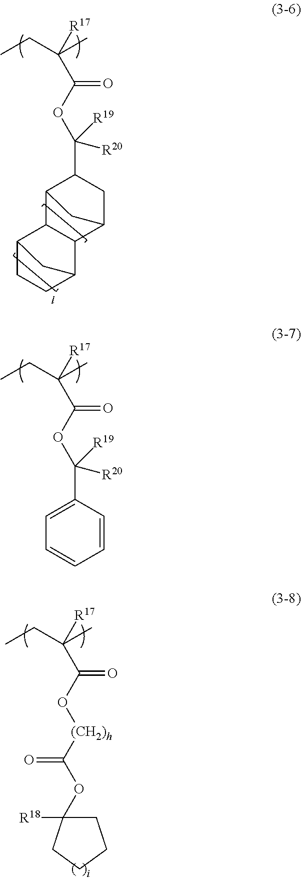

In the formulas (3-1) to (3-8), R¹⁷ to R²⁰ have the same meaning as in the formula (3), h is an integer of 1 to 4, i and j are each independently an integer of 1 to 4, and k and 1 are each 0 or 1.

In the formulas (3-1) to (3-8), i and j are preferably 1, and R¹⁸ is preferably a methyl group, an ethyl group, an isopropyl group, t-butyl group, a cyclopentyl group, or a phenyl group. R¹⁹ and R²⁰ are each preferably a methyl group, or an ethyl group

The structural unit (I-1) is preferably a structural unit (I-1-1) represented by the formula (3-1). In this case, R¹⁸ is preferably an isopropyl group, a t-butyl group, or a phenyl group.

The base polymer may contain one type or a combination of two or more types of the structural units (I).

The lower limit of the content by percent of the structural unit (I) (a total content by percent when a plurality of types are contained) is preferably 10 mol %, more preferably 20 mol %, still more preferably 30 mol %, and particularly preferably 40 mol % based on all structural units constituting the base polymer. The upper limit of the content by percent is preferably 80 mol %, more preferably 70 mol %, still more preferably 60 mol %, and particularly preferably 55 mol %. When the content of the structural unit (I) is set to fall within the above range, the pattern-forming performance of the radiation-sensitive composition can further be improved.

### [Structural Unit (II)]

The structural unit (II) is a structural unit including at least one selected from the group consisting of a lactone structure, a cyclic carbonate structure and a sultone structure. The solubility of the base polymer into a developer can be adjusted by further introducing the structural unit (II). As a result, the radiation-sensitive composition can provide improved lithography properties such as the resolution. The adhesion between a resist pattern formed from the base polymer and a substrate can also be improved.

Examples of the structural unit (II) include structural units represented by the formulae (T-1) to (T-11).

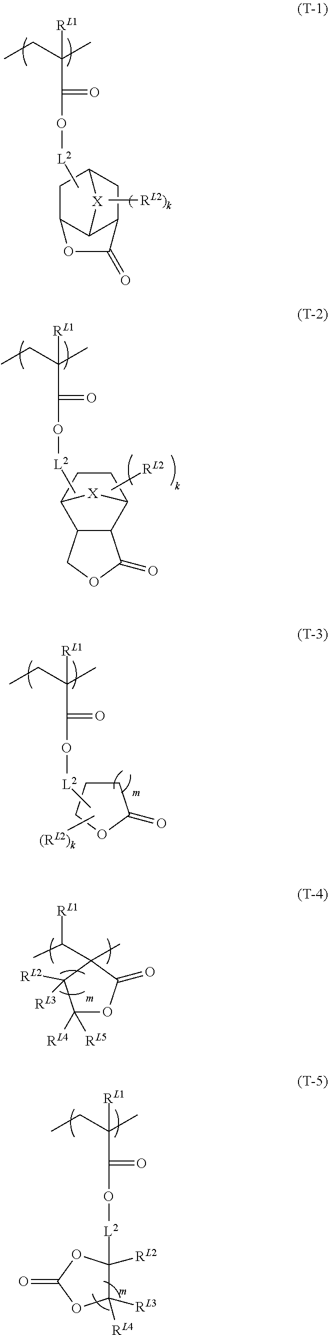

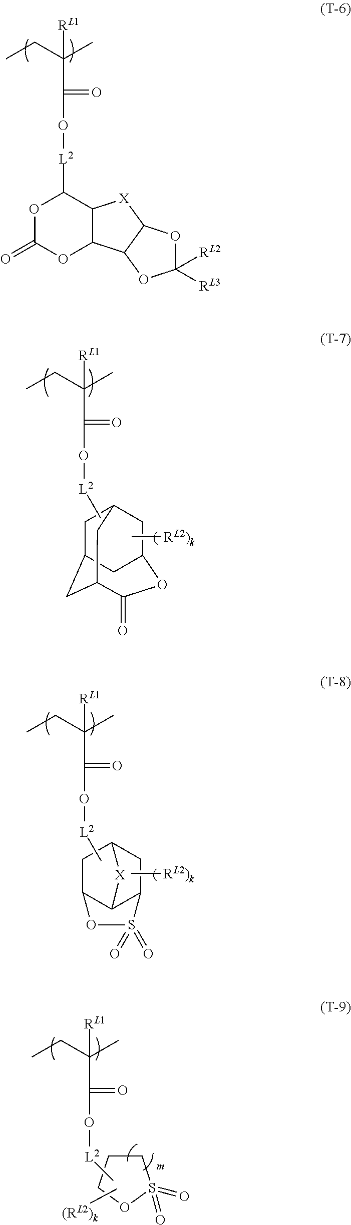

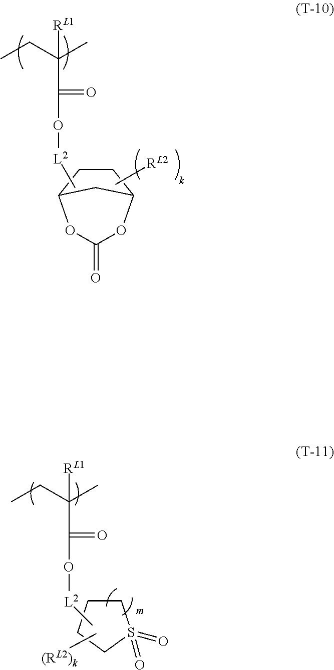

In the formulae, R^{L1} is a hydrogen atom, a fluorine atom, a methyl group, or a trifluoromethyl group; R^{L2} to R^{L5} are each independently a hydrogen atom, an alkyl group having a carbon number of 1 to 4, a cyano group, a trifluoromethyl group, a methoxy group, a methoxycarbonyl group, a hydroxy group, a hydroxymethyl group, or a dimethylamino group; R^{L4} and R^{L5} may be a divalent alicyclic group having a carbon number of 3 to 8, which is obtained by combining R^{L4} and R^{L5} with the carbon atom to which they are bound. L² is a single bond, or a divalent linking group; X is an oxygen atom or a methylene group; k is an integer of 0 to 3; and m is an integer of 1 to 3.

Example of the divalent alicyclic group having a carbon number of 3 to 8, which is composed of a combination of R^{L4} and R^{L5} with the carbon atom to which they are bound, includes the divalent alicyclic group having a carbon number of 3 to 8 in the divalent alicyclic group having a carbon number of 3 to 20, which is formed by a combination of R¹⁹ and R²⁰ in the formula (3) with the carbon atom to which they are bound. One or more hydrogen atoms on the alicyclic group may be substituted with a hydroxy group.

Examples of the divalent linking group represented by L² as described above include a divalent straight or branched chain hydrocarbon group having a carbon number of 1 to 10; a divalent alicyclic hydrocarbon group having a carbon number of 4 to 12; and a group composed of one or more of the hydrocarbon group thereof and at least one group of —CO—, —O—, —NH— and —S—.

Among them, the structural unit (II) is preferably a group having a lactone structure, more preferably a group having a norbornane lactone structure, and further preferably a group derived from a norbornane lactone-yl (meth)acrylate.

The lower limit of the content by percent of the structural unit (II) (a total content by percent when a plurality of types are contained) is preferably 10 mol %, more preferably 20 mol %, and still more preferably 30 mol % based on all structural units constituting the base polymer. The upper limit of the content by percent is preferably 80 mol %, more preferably 70 mol %, and still more preferably 60 mol %. By adjusting the content by percent of the structural unit D within the ranges, the radiation-sensitive composition can provide improved lithography properties such as the resolution. The adhesion between the formed resist pattern and the substrate can also be improved.

### [Structural Unit (III)]

The base polymer optionally has another structural unit in addition to the structural units (I) and (II). Another structural unit includes a structural unit (III) containing a polar group (excluding those corresponding to the structural unit (II)). When the base polymer further has a structural unit (III), solubility in the developer can be adjusted. As a result, lithographic performance such as resolution of the radiation-sensitive composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit (III) include structural units represented by the formulas.

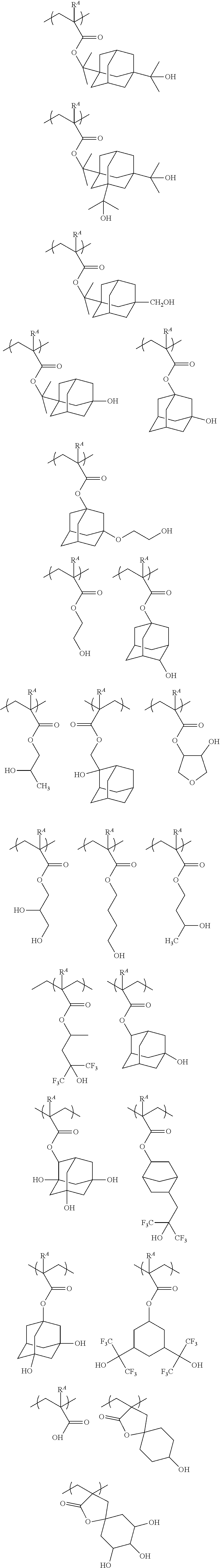

In the formulas, R^{A} is a hydrogen atom, a fluorine atom, a methyl group, or a trifluoromethyl group.

When the base polymer has the structural unit (III) having a polar group, the lower limit of the content by percent of the structural unit (III) (a total content by percent when a plurality of types are contained) is preferably 3 mol %, more preferably 5 mol %, and still more preferably 8 mol % based on all structural units constituting the base polymer. The upper limit of the content by percent is preferably 40 mol %, more preferably 30 mol %, and still more preferably 25 mol %. When the content of the structural unit having a polar group is set to fall within the above range, the radiation-sensitive composition can provide further improved lithography properties such as the resolution.

### [Structural Unit (IV)]

The base polymer optionally has, as another structural unit, a structural unit having a phenolic hydroxyl group (hereinafter, also referred to as “structural unit (IV)”), in addition to the structural unit (III) having a polar group. The structural unit (IV) contributes to an improvement in etching resistance and an improvement in a difference in solubility of a developer (dissolution contrast) between an exposed part and a non-exposed part. In particular, the structural unit (IV) can be suitably applied to pattern formation using exposure with a radioactive ray having a wavelength of 50 nm or less, such as an electron beam or EUV. In this case, the polymer preferably has the structural unit (I) together with the structural unit (IV).

The structural unit containing a phenolic hydroxy group is represented by, for example, the formulas (4-1) to (4-6).

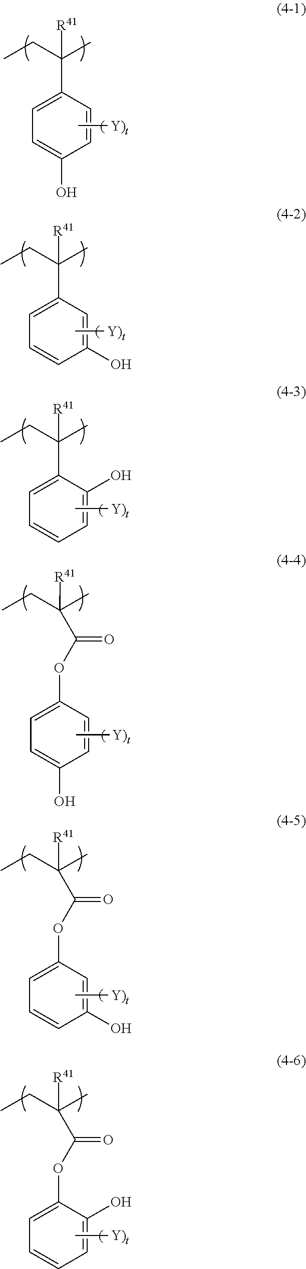

In the formulas (4-1) to (4-6), R⁴¹ is independently at each occurrence a hydrogen atom, a fluorine atom, a methyl group, or a trifluoromethyl group. Y is a halogen atom, a trifluoromethyl group, a cyano group, an alkyl or alkoxy group having 1 to 6 carbon atoms, or an acyl, acyloxy, or alkoxycarbonyl group having 2 to 7 carbon atoms. When there are a plurality of Y's, the plurality of Y's are the same or different from each other, t is an integer of 0 to 4.

When the structural unit (IV) is obtained, it is preferable to obtain the structural unit (IV) by polymerizing the monomer in a state where the phenolic hydroxy group is protected by a protecting group such as an alkali-dissociable group (e.g., an acyl group) during polymerization, and then deprotecting the polymerized product by hydrolysis. Alternatively, the polymerization of a monomer that gives the structural unit (IV) may be carried out without protecting the phenolic hydroxyl group.

In the case of a polymer for exposure to radiation having a wavelength of 50 nm or less, the lower limit of the content by percent of the structural unit (IV) is preferably 10 mol %, and more preferably 20 mol % based on all structural units constituting the polymer. The upper limit of the content by percent is preferably 70 mol %, and more preferably 60 mol %.

### [Other Structural Unit]

The base polymer may contain, as a structural unit other than the structural units listed above, a structural unit represented by the formula (6) and containing an alicyclic structure (hereinafter, also referred to as “structural unit (VII)”).

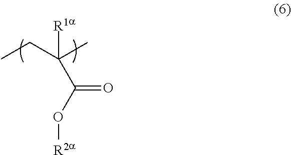

In the formula (6), Ria represents a hydrogen atom, a fluorine atom, a methyl group, or a trifluoromethyl group, and R^{2a} represents a monovalent alicyclic hydrocarbon group having 3 to 20 carbon atoms.

In the formula (6), as the monovalent alicyclic hydrocarbon group having 3 to 20 carbon atoms represented by R^{2a}, the monovalent alicyclic hydrocarbon groups having 3 to 20 carbon atoms indicated as W in the formula (1) can be suitably employed.

When the base polymer contains the structural unit (VII), the lower limit of the content by percent of the structural unit (VII) is preferably 2 mol, more preferably 5 mol, and still more preferably 8 mol % based on all structural units constituting the base polymer. The upper limit of the content by percent is preferably 40 mol %, more preferably 30 mol %, and still more preferably 20 mol %.

### (Synthesis Method of Base Polymer)

For example, the base polymer can be synthesized by performing a polymerization reaction of each monomer for providing each structural unit with a radical polymerization initiator or the like in a suitable solvent.

Examples of the radical polymerization initiator include an azo-based radical initiator, including azobisisobutyronitrile (AIBN), 2,2′-azobis(4-methoxy-2,4-dimethylvaleronitrile), 2,2′-azobis(2-cyclopropylpropanenitrile), 2,2′-azobis(2,4-dimethylvaleronitrile), and dimethyl 2,2′-azobisisobutyrate; and peroxide-based radical initiator, including benzoyl peroxide, t-butyl hydroperoxide, and cumene hydroperoxide. Among them, AIBN or dimethyl 2,2′-azobisisobutyrate is preferred, and AIBN is more preferred. The radical initiator may be used alone, or two or more radical initiators may be used in combination.

Examples of the solvent used for the polymerization reaction include
alkanes including n-pentane, n-hexane, n-heptane, n-octane, n-nonane, and n-decane;
cycloalkanes including cyclohexane, cycloheptane, cyclooctane, decalin, and norbornane;
aromatic hydrocarbons including benzene, toluene, xylene, ethylbenzene, and cumene;
halogenated hydrocarbons including chlorobutanes, bromohexanes, dichloroethanes, hexamethylenedibromide, and chlorobenzenes;
saturated carboxylate esters, including ethyl acetate, n-butyl acetate, i-butyl acetate, and methyl propionate;
cyclic esters such as γ-butyrolactone;
ketones including acetone, methyl ethylketone, 2-butanone, 4-methyl-2-pentanone, and 2-heptanone;
ethers including tetrahydrofuran, dimethoxyethanes, diethoxyethanes, and polyhydric alcohol partial ethers; and
alcohols including methanol, ethanol, 1-propanol, 2-propanol, and 4-methyl-2-pentanol. The solvent used for the polymerization reaction may be used alone, or two or more solvents may be used in combination.

The reaction temperature of the polymerization reaction is typically from 40° C. to 150° C., and preferably from 50° C. to 120° C. The reaction time is typically from 1 hour to 48 hours, and preferably from 1 hour to 24 hours.

The molecular weight of the base polymer is not particularly limited, and the lower limit of the weight-average molecular weight (Mw) equivalent to polystyrene determined by gel permeation chromatography (GPC) is preferably 3,000, more preferably 4,000, still more preferably 5,000, and particularly preferably 6,000. The upper limit of the Mw is preferably 20,000, more preferably 15,000, still more preferably 10,000, and particularly preferably 8,000. By setting the Mw of the base polymer within the above range, the resulting resist film can exhibit good heat resistance and developability.

For the base polymer as a base polymer, the ratio of Mw to the number average molecular weight (Mn) as determined by GPC relative to standard polystyrene (Mw/Mn) is typically not less than 1 and not more than 5, preferably not less than 1 and not more than 3, and more preferably not less than 1 and not more than 2.

The Mw and Mn of the polymer in the specification are amounts measured by using Gel Permeation Chromatography (GPC) with the condition as described below.
GPC column: two G2000HXL, one G3000HXL, and one G4000HXL (all manufactured from Tosoh Corporation)
Column temperature: 40° C.
Eluting solvent: tetrahydrofuran
Flow rate: 1.0 mL/min
Sample concentration: 1.0% by mass
Sample injection amount: 100 μL
Detector: Differential Refractometer
Reference material: monodisperse polystyrene

The content by percent of the base polymer is preferably 60% by mass or more, more preferably 65% by mass or more, and still more preferably 70% by mass or more based on the total solid content of the radiation-sensitive composition.

### (Another Polymer)

The radiation-sensitive composition according to the present embodiment may contain, as another polymer, a polymer having higher content by mass of fluorine atoms than the above-described base polymer (hereinafter, also referred to as a “high fluorine-content polymer”). When the radiation-sensitive composition contains the high fluorine-content polymer, the high fluorine-content polymer can be localized in the surface layer of a resist film compared to the base polymer, which as a result makes it possible to enhance the water repellency of the surface of the resist film during immersion exposure or to perform surface modification of the resist film during EUV exposure or control of the distribution of the composition in the film.

The high fluorine-content polymer preferably has, for example, a structural unit represented by the formula (5) (hereinafter, also referred to as “structural unit (V)”), and may have the structural unit (I) or the structural unit (III) in the base polymer as necessary.

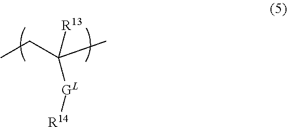

In the formula (5), R¹³ is a hydrogen atom, a methyl group, or a trifluoromethyl group; G^{L} is a single bond, an alkanediyl group having 1 to 5 carbon atoms, an oxygen atom, a sulfur atom, —COO—, —SO₂ONH—, —CONH—, —OCONH—, or a combination thereof; and R¹⁴ is a monovalent fluorinated chain hydrocarbon group having a carbon number of 1 to 20, or a monovalent fluorinated alicyclic hydrocarbon group having a carbon number of 3 to 20.

As R¹³ as described above, in terms of the copolymerizability of monomers resulting in the structural unit (V), a hydrogen atom or a methyl group is preferred, and a methyl group is more preferred.

As G^{L} as described above, in terms of the copolymerizability of monomers resulting in the structural unit (V), a single bond or —COO— is preferred, and —COO— is more preferred.

Example of the monovalent fluorinated chain hydrocarbon group having a carbon number of 1 to 20 represented by R¹⁴ as described above includes a group in which a part of or all of hydrogen atoms in the straight or branched chain alkyl group having a carbon number of 1 to 20 is/are substituted with a fluorine atom.

Example of the monovalent fluorinated alicyclic hydrocarbon group having a carbon number of 3 to 20 represented by R¹⁴ as described above includes a group in which a part of or all of hydrogen atoms in the monocyclic or polycyclic hydrocarbon group having a carbon number of 3 to 20 is/are substituted with a fluorine atom.

The R¹⁴ as described above is preferably a fluorinated chain hydrocarbon group, more preferably a fluorinated alkyl group, and further preferably 2,2,2-trifluoroethyl group, 2,2,3,3,3-pentafluoropropyl group, 1,1,1,3,3,3-hexafluoropropyl group and 5,5,5-trifluoro-1,1-diethylpentyl group.

When the high fluorine-content polymer has the structural unit (V), the lower limit of the content by percent of the structural unit (V) is preferably 50 mol %, more preferably 60 mol %, and still more preferably 70 mol % based on the total amount of all structural units constituting the high fluorine-content polymer. The upper limit of the content by percent is preferably 95 mol %, more preferably 90 mol %, and still more preferably 85 mol %. When the content of the structural unit (V) is set to fall within the above range, the content by mass of fluorine atoms of the high fluorine-content polymer can more appropriately be adjusted to further promote the localization of the high fluorine-content polymer in the surface layer of a resist film, as a result, the water repellency of the resist film during immersion exposure or adjustment of the quality of resist film during EUV exposure can be further improved.

The high fluorine-content polymer may have a fluorine atom-containing structural unit represented by the formula (f-2) (hereinafter, also referred to as a “structural unit (VI)”) in addition to or in place of the structural unit (V). When the high fluorine-content polymer has the structural unit (VI), solubility in an alkaline developing solution is improved, and therefore generation of development defects can be prevented.

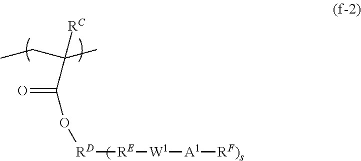

The structural unit (VI) is classified into two groups: a unit having an alkali soluble group (x); and a unit having a group (y) in which the solubility into the alkaline developing solution is increased by the dissociation by alkali (hereinafter, simply referred as an “alkali-dissociable group”). In both cases of (x) and (y), R^{C} in the formula (f-2) is a hydrogen atom, a fluorine atom, a methyl group, or a trifluoromethyl group; R^{D} is a single bond, a hydrocarbon group having a carbon number of 1 to 20 with the valency of (s+1), a structure in which an oxygen atom, a sulfur atom, —NR^{dd}—, a carbonyl group, —COO—, —OCO—, or —CONH— is connected to the terminal on R^{E} side of the hydrocarbon group, or a structure in which a part of hydrogen atoms in the hydrocarbon group is substituted with an organic group having a hetero atom; R^{dd} is a hydrogen atom, or a monovalent hydrocarbon group having a carbon number of 1 to 10; and s is an integer of 1 to 3.

When the structural unit (VI) has the alkali soluble group (x), R^{F} is a hydrogen atom; A¹ is an oxygen atom, —COO—* or —SO₂O—*; * refers to a bond to R^{F}; W¹ is a single bond, a hydrocarbon group having a carbon number of 1 to 20, or a divalent fluorinated hydrocarbon group. When A¹ is an oxygen atom, W¹ is a fluorinated hydrocarbon group having a fluorine atom or a fluoroalkyl group on the carbon atom connecting to A¹. R^{E} is a single bond, or a divalent organic group having a carbon number of 1 to 20. When s is 2 or 3, a plurality of R^{E}, W¹, A¹ and R^{F} may be each identical or different. The affinity of the high fluorine-content polymer into the alkaline developing solution can be improved by including the structural unit (VI) having the alkali soluble group (x), and thereby prevent from generating the development defect. As the structural unit (VI) having the alkali soluble group (x), particularly preferred is a structural unit in which A¹ is an oxygen atom and W¹ is a 1,1,1,3,3,3-hexafluoro-2,2-methanediyl group.

When the structural unit (VI) has the alkali-dissociable group (y), R^{F} is a monovalent organic group having carbon number of 1 to 30; A¹ is an oxygen atom, —NR^{aa}—, —COO—*, —OCO—*, or —SO₂O—*; R^{aa} is a hydrogen atom, or a monovalent hydrocarbon group having a carbon number of 1 to 10; * refers to a bond to R^{F}; W¹ is a single bond, or a divalent fluorinated hydrocarbon group having a carbon number of 1 to 20; R^{E} is a single bond, or a divalent organic group having a carbon number of 1 to 20. When A¹ is —COO—*, —OCO—* or —SO₂O—*, W¹ or R^{F} has a fluorine atom on the carbon atom connecting to A¹ or on the carbon atom adjacent to the carbon atom. When A¹ is an oxygen atom, W¹ and R^{E} are a single bond; R^{D} is a structure in which a carbonyl group is connected at the terminal on R^{E} side of the hydrocarbon group having a carbon number of 1 to 20; and R^{F} is an organic group having a fluorine atom. When s is 2 or 3, a plurality of R^{E}, W¹, A¹ and R^{F} may be each identical or different. The surface of the resist film is changed from hydrophobic to hydrophilic in the alkaline developing step by including the structural unit (VI) having the alkali-dissociable group (y). As a result, the affinity of the high fluorine-content polymer into the alkaline developing solution can be significantly improved, and thereby prevent from generating the development defect more efficiently. As the structural unit (VI) having the alkali-dissociable group (y), particularly preferred is a structural unit in which A¹ is —COO—*, and R^{F} or W¹, or both is/are a fluorine atom.

In terms of the copolymerizability of monomers resulting in the structural unit (VI), R^{C} is preferably a hydrogen atom or a methyl group, and more preferably a methyl group.

When R^{E} is a divalent organic group, R^{E} is preferably a group having a lactone structure, more preferably a group having a polycyclic lactone structure, and further preferably a group having a norbornane lactone structure.

When the high fluorine-content polymer has the structural unit (VI), the lower limit of the content by percent of the structural unit (VI) is preferably 40 mol %, more preferably 50 mol %, and still more preferably 55 mol % based on the total amount of all structural units constituting the high fluorine-content polymer. The upper limit of the content by percent is preferably 95 mol %, more preferably 90 mol %, and still more preferably 85 mol %. When the content ratio of the structural unit (VI) is set to fall within the above range, the water repellency of the resist film at the time of immersion exposure can be enhanced, and the solubility in an alkaline developer can be improved to suppress the occurrence of development defects.

### [Other Structural Unit]

The high fluorine-content polymer may contain a structural unit (VII) in addition to the structural unit (I) and the structural unit (III) in the base polymer as a structural unit other than the structural units listed above.

When the high fluorine-content polymer contains the structural unit (I), the structural unit (III), and the structural unit (VII), the content ratio described for the base polymer can be suitably employed as the content ratio of each of the structural units in the high fluorine-content polymer.

The lower limit of the Mw of the high fluorine-content polymer is preferably 2,000, more preferably 3,000, still more preferably 4,000, and particularly preferably 5,000. The upper limit of the Mw is preferably 20,000, more preferably 15,000, still more preferably 10,000, particularly preferably 8,000.

The lower limit of the Mw/Mn of the high fluorine-content polymer is usually 1, and more preferably 1.1. In addition, the upper limit of the Mw/Mn is usually 5, preferably 3, and more preferably 2.

When the radiation-sensitive composition contains the high-fluorine-content polymer, the content of the high fluorine-containing polymer is preferably 0.5 parts by mass or more, more preferably 1 part by mass or more, still more preferably 1.5 parts by mass or more, and particularly preferably 2 parts by mass or more based on 100 parts by mass of the base polymer. The content of the high fluorine-containing polymer is preferably 15 parts by mass or less, more preferably 10 parts by mass or less, still more preferably 8 parts by mass or less, and particularly preferably 6 parts by mass or less.

When the content of the high fluorine-content polymer is set to fall within the above range, the high fluorine-content polymer can more effectively be localized in the surface layer of a resist film, which as a result makes it possible to further enhance the water repellency of the surface of the resist film during immersion exposure. Further, it is possible to highly control surface modification of the resist film during EUV exposure or control of the distribution of the composition in the film. The radiation-sensitive composition may contain one kind of high fluorine-content polymer or two or more kinds of high fluorine-content polymers.

### (Method for Synthesizing High Fluorine-Content Polymer)

The high fluorine-content polymer can be synthesized by a method similar to the above-described method for synthesizing a base polymer.

### (Acid Diffusion Controlling Agent)

The radiation-sensitive composition may include an acid diffusion controlling agent, if needed. The acid diffusion controlling agent has an effect of controlling the diffusion phenomenon in which an acid resulted from the onium salt compound (1) by the exposure is diffused in the resist film, and of inhibiting undesired chemical reaction in the non-exposed part. The acid diffusion controlling agent can also improve the storage stability of the resulting radiation-sensitive composition. The acid diffusion controlling agent can further improve the resolution of the resist pattern and prevent from changing the line width of the resist pattern because of the variation of the pulling and placing time, i.e., the time from the exposure to the developing treatment, and therefore provide the radiation-sensitive composition having an improved process stability.

Examples of the acid diffusion controlling agent include a compound represented by the formula (7) (hereinafter, also referred as a “nitrogen-containing compound (I)”); a compound having two nitrogen atoms in one molecule (hereinafter, also referred as a “nitrogen-containing compound (II)”); a compound having three nitrogen atoms in one molecule (hereinafter, also referred as a “nitrogen-containing compound (III)”); a compound having an amide group; a urea compound; and a nitrogen-containing heterocyclic ring compound.

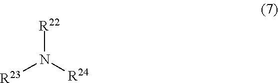

In the formula (7), R²², R²³ and R²⁴ are each independently a hydrogen atom, a substituted or unsubstituted alkyl group, a substituted or unsubstituted cycloalkyl group, a substituted or unsubstituted aryl group, or a substituted or unsubstituted aralkyl group.

Examples of the nitrogen-containing compound (I) include a monoalkylamine including n-hexylamine; a dialkylamine including di-n-butylamine; a trialkylamine including triethylamine; and an aromatic amine including aniline, 2,6-diisopropylaniline.

Examples of the nitrogen-containing compound (II) include ethylenediamine and N,N,N′,N′-tetramethylethylenediamine.

Examples of the nitrogen-containing compound (III) include a polyamine compound, including polyethyleneimine and polyallylamine; and a polymer including dimethylaminoethylacrylamide.

Examples of the amide-containing compound include formamide, N-methylformamide, N,N-dimethylformamide, acetamide, N-methylacetamide, N,N-dimethylacetamide, propionamide, benzamide, pyrrolidone, and N-methyl pyrrolidone.

Examples of the urea compound include urea, methylurea, 1,1-dimethylurea, 1,3-dimethylurea, 1,1,3,3-tetramethylurea, 1,3-diphenylurea, and tributylthiourea.

Examples of the nitrogen-containing heterocyclic ring compound include pyridines, including pyridine and 2-methylpyridine; morpholines, including N-propylmorpholine and N-(undecylcarbonyloxyethyl)morpholine; pyrazine, and pyrazole.

A compound having an acid-dissociable group may be used as the nitrogen-containing organic compound. Examples of the nitrogen-containing organic compound having an acid-dissociable group include N-t-butoxycarbonylpiperidine, N-t-butoxycarbonylimidazole, N-t-butoxycarbonylbenzimidazole, N-t-butoxycarbonyl-2-phenylbenzimidazole, N-(t-butoxycarbonyl)di-n-octylamine, N-(t-butoxycarbonyl)diethanolamine, N-(t-butoxycarbonyl)dicyclohexylamine, N-(t-butoxycarbonyl)diphenylamine, N-t-butoxycarbonyl-4-hydroxypiperidine, N-t-butoxycarbonyl-4-acetoxypiperidine, and N-t-amyloxycarbonyl-4-hydroxypiperidine.

As the acid diffusion control agent, a radiation-sensitive weak acid generator that generates a weak acid upon exposure can also be preferably used. The acid generated from the radiation-sensitive weak acid generator is a weak acid that does not induce the dissociation of the acid-dissociable groups in the polymer under the conditions for dissociating the acid-dissociable groups. In the present specification, “dissociation” of an acid-dissociable group means dissociation upon post-exposure baking at 110° C. for 60 seconds.

Example of the radiation-sensitive weak acid generator includes an onium salt compound in which the compound is degraded by the exposure to lose the acid diffusion controlling properties. Examples of the onium salt compound include a sulfonium salt compound represented by the formula (8-1), and an iodonium salt compound represented by the formula (8-2). In addition, a compound represented by the formula (8-3) containing a sulfonium cation and an anion in the same molecule and a compound represented by the formula (8-4) containing an iodonium cation and an anion in the same molecule are also included.

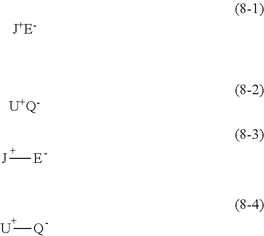

In the formulae (8-1) to formula (8-4), J⁺ is a sulfonium cation; and U⁺ is an iodonium cation. Examples of the sulfonium cation represented by J⁺ include sulfonium cations represented by the formulae (X-1) to (X-4). Examples of the iodonium cation represented by U⁺ include iodonium cations represented by the formulae (X-5) to (X-6). E⁻ and Q⁻ are each independently anion represented by OH⁻, R^{α}—COO⁻, and R^{α}—SO₃. R^{α} is a single bond or a monovalent organic group having 1 to 30 carbon atoms. Examples of the organic group include a monovalent hydrocarbon group having 1 to 20 carbon atoms, a group having a divalent hetero atom-containing group between carbon and carbon or at a carbon chain end of the hydrocarbon group, a group obtained by substituting some or all of hydrogen atoms of the hydrocarbon group with a monovalent hetero atom-containing group, or a combination thereof.

As the monovalent hydrocarbon group having 1 to 20 carbon atoms, a monovalent hydrocarbon group having 1 to 20 carbon atoms represented by R^{A1} and R^{A2} in the formulas (A-3) and (A-4) can be suitably employed.

Examples of hetero atoms that constitute the divalent or monovalent hetero atom-containing group include an oxygen atom, a nitrogen atom, a sulfur atom, a phosphorus atom, a silicon atom, and a halogen atom. Examples of the halogen atom include a fluorine atom, a chlorine atom, a bromine atom, and an iodine atom.

Examples of the divalent hetero atom-containing group include —CO—, —CS—, —NH—, —O—, —S—, —SO—, —SO₂—, and a group obtained by combining these.

Examples of the monovalent hetero atom-containing group include a hydroxy group, a sulfanyl group, a cyano group, a nitro group, and s halogen atom.

Examples of the radiation-sensitive weak acid generator include compounds represented by the formulae.

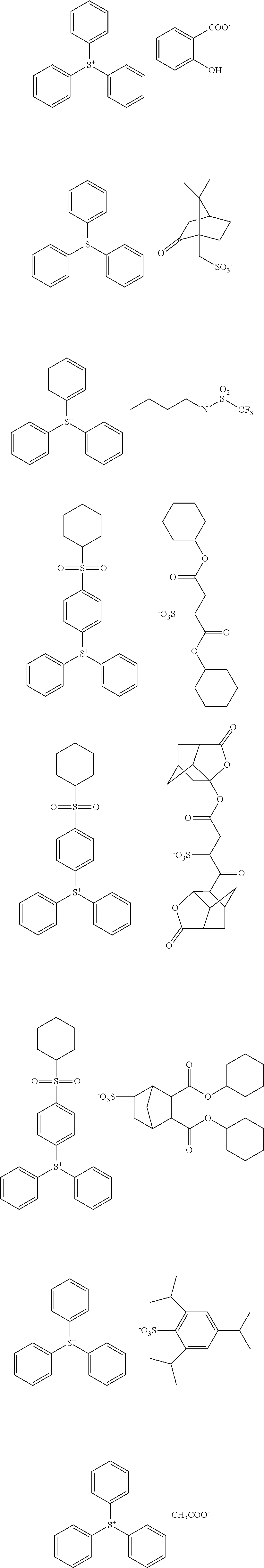

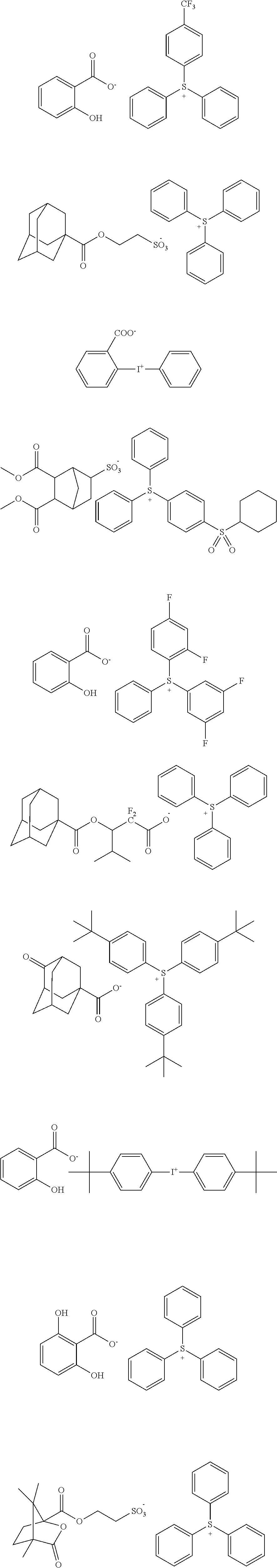

Among them, the radiation-sensitive weak acid generator is preferably the sulfonium salt, more preferably a triarylsulfonium salt, and further preferably a triphenylsulfonium salicylate or triphenylsulfonium 10-camphorsulfonate.

The lower limit of the content of the acid diffusion controlling agent is preferably 0.5 parts by mass, more preferably 1 part by mass, and still more preferably 2 parts by mass based on 100 parts by mass of the polymer. The upper limit of the content is preferably 30 parts by mass, more preferably 20 parts by mass, and still more preferably 15 parts by mass.

By adjusting the content of the acid diffusion controlling agent within the ranges, the radiation-sensitive composition can provide improved lithography properties. The radiation-sensitive composition may contain one type of the acid diffusion controlling agent, or two or more acid diffusion controlling agents in combination.

### (Solvent)

The radiation-sensitive composition according to the present embodiment contains a solvent. The solvent is not particularly limited as long as it can dissolve or disperse at least the onium salt compound (1), the polymer, and an acid diffusion controlling agent or the like contained as necessary.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
a monoalcohol-based solvent having a carbon number of 1 to 18, including iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
a polyhydric alcohol having a carbon number of 2 to 18, including ethylene glycol, 1,2-propylene glycol, 2-methyl-2,4-pentanediol, 2,5-hexanediol, diethylene glycol, dipropylene glycol, triethylene glycol, and tripropylene glycol; and
a partially etherized polyhydric alcohol-based solvent in which a part of hydroxy groups in the polyhydric alcohol-based solvent is etherized.

In the present embodiment, the alcohol-based solvents also include alcohol acid ester-based solvents such as methyl lactate, ethyl lactate, propyl lactate, butyl lactate, methyl 2-hydroxyisobutyrate, i-propyl 2-hydroxyisobutyrate, i-butyl 2-hydroxyisobutyrate, and n-butyl 2-hydroxyisobutyrate.

Examples of the ether-based solvent include:
a dialkyl ether-based solvent, including diethyl ether, dipropyl ether, and dibutyl ether;
a cyclic ether-based solvent, including tetrahydrofuran and tetrahydropyran;
an ether-based solvent having an aromatic ring, including diphenylether and anisole (methyl phenyl ether); and
an etherized polyhydric alcohol-based solvent in which a hydroxy group in the polyhydric alcohol-based solvent is etherized.

Examples of the ketone-based solvent include:
a chain ketone-based solvent, including acetone, butanone, and methyl-iso-butyl ketone;
a cyclic ketone-based solvent, including cyclopentanone, cyclohexanone, and methylcyclohexanone; and
2,4-pentanedione, acetonylacetone, and acetophenone.

Examples of the amide-based solvent include:
a cyclic amide-based solvent, including N,N′-dimethyl imidazolidinone and N-methylpyrrolidone; and
a chain amide-based solvent, including N-methylformamide, N,N-dimethylformamide, N,N-diethylformamide, acetamide, N-methylacetamide, N,N-dimethylacetamide, and N-methylpropionamide.

Examples of the ester-based solvent include:
a monocarboxylate ester-based solvent, including n-butyl acetate and ethyl lactate;
a partially etherized polyhydric alcohol acetate-based solvent, including diethylene glycol mono-n-butyl ether acetate, propylene glycol monomethyl ether acetate, and dipropylene glycol monomethyl ether acetate;
a lactone-based solvent, including γ-butyrolactone and valerolactone;
a carbonate-based solvent, including diethyl carbonate, ethylene carbonate, and propylene carbonate; and
a polyhydric carboxylic acid diester-based solvent, including propylene glycol diacetate, methoxy triglycol acetate, diethyl oxalate, ethyl acetoacetate, ethyl lactate, and diethyl phthalate.

Examples of the hydrocarbon-based solvent include:
an aliphatic hydrocarbon-based solvent, including n-hexane, cyclohexane, and methylcyclohexane;
an aromatic hydrocarbon-based solvent, including benzene, toluene, di-iso-propylbenzene, and n-amylnaphthalene.

Among them, an alcohol-based solvent, an ester-based solvent and an ether-based solvent are preferable, an alcohol acid ester-based solvent, a polyhydric alcohol partial ether acetate-based solvent, a lactone-based solvent, a monocarboxylic acid ester-based solvent, and a ketone-based solvent are more preferable, and propylene glycol monomethyl ether acetate, γ-butyrolactone, ethyl lactate, and cyclohexanone are still more preferable. The radiation-sensitive composition may include one type of the solvent, or two or more types of the solvents in combination.

### <Other Optional Components>

The radiation-sensitive composition may contain other optional components other than the above-descried components. Examples of other optional components include a cross-linking agent, a localization enhancing agent, a surfactant, an alicyclic backbone-containing compound, and a sensitizer. These other optional components may be used singly or in combination of two or more of them.

### <Method for Preparing Radiation-Sensitive Composition>

The radiation-sensitive composition can be prepared by, for example, mixing the onium salt compound (1), the polymer, and, as necessary, the acid diffusion controlling agent or the like, as well as the solvent in a prescribed ratio. The radiation-sensitive composition is preferably filtered through, for example, a filter having a pore diameter of about 0.05 μm to 0.40 μm after mixing. The solid matter concentration of the radiation-sensitive composition is usually 0.1 mass % to 50 mass %, preferably 0.5 mass % to 30 mass %, more preferably 1 mass % to 20 mass %.

### <Method for Forming Pattern>

A pattern forming method according to an embodiment of the present disclosure includes:
applying the radiation-sensitive composition directly or indirectly on a substrate to form a resist film (hereinafter, also referred to as a “resist film forming step”);
exposing the resist film (hereinafter, also referred to as an “exposure step”); and
developing the exposed resist film (hereinafter, also referred to as a “developing step”).

According to the method for forming a pattern, since the radiation-sensitive composition capable of exhibiting excellent sensitivity, LWR performance, and CDU performance in pattern formation, and having good storage stability is used, a high-quality resist pattern can be efficiently formed. Hereinbelow, each of the steps will be described.

### [Resist Film Forming Step]

In this step, a resist film is formed with the radiation-sensitive composition. Examples of the substrate on which the resist film is formed include one traditionally known in the art, including a silicon wafer, silicon dioxide, and a wafer coated with aluminum. An organic or inorganic antireflection film may be formed on the substrate, as disclosed in JP-B-06-12452 and JP-A-59-93448. Examples of the applicating method include a rotary coating (spin coating), flow casting, and roll coating. After applicating, a prebake (PB) may be carried out in order to evaporate the solvent in the film, if needed. The temperature of PB is typically from 70° C. to 150° C., and preferably from 90° C. to 140° C. The duration of PB is typically from 5 seconds to 600 seconds, and preferably from 10 seconds to 300 seconds.

The lower limit of the thickness of the resist film to be formed is preferably 10 nm, more preferably 15 nm, and still more preferably 20 nm. The upper limit of the film thickness is preferably 500 nm, more preferably 400 nm, and still more preferably 300 nm. In particular, when a thick resist film is exposed to ArF excimer laser light in an exposure step described later, the lower limit of the film thickness may be 100 nm, may be 150 nm, or may be 200 nm.

When the immersion exposure is carried out, irrespective of presence of a water repellent polymer additive such as the high fluorine-content polymer in the radiation-sensitive composition, the formed resist film may have a protective film for the immersion which is not soluble into the immersion liquid on the film in order to prevent a direct contact between the immersion liquid and the resist film. As the protective film for the immersion, a solvent-removable protective film that is removed with a solvent before the developing step (for example, see JP-A-2006-227632); or a developer-removable protective film that is removed during the development of the developing step (for example, see WO2005-069076 and WO2006-035790) may be used. In terms of the throughput, the developer-removable protective film is preferably used.

When the next step, the exposure step, is performed with radiation having a wavelength of 50 nm or less, it is preferable to use a polymer having the structural unit (I) and the structural unit (IV) as the base polymer in the composition.

### [Exposing Step]

In this step, the resist film formed in the resist film forming step as the step (1) is exposed by irradiating with a radioactive ray through a photomask (optionally through an immersion medium such as water). Examples of the radioactive ray used for the exposure include visible ray, ultraviolet ray, far ultraviolet ray, extreme ultraviolet ray (EUV); an electromagnetic wave including X ray and γ ray; an electron beam; and a charged particle radiation such as α ray. Among them, far ultraviolet ray, an electron beam, or EUV is preferred. ArF excimer laser light (wavelength is 193 nm), KrF excimer laser light (wavelength is 248 nm), an electron beam, or EUV is more preferred. An electron beam or EUV having a wavelength of 50 nm or less which is identified as the next generation exposing technology is further preferred.

When the exposure is carried out by immersion exposure, examples of the immersion liquid include water and fluorine-based inert liquid. The immersion liquid is preferably a liquid which is transparent with respect to the exposing wavelength, and has a minimum temperature factor of the refractive index so that the distortion of the light image reflected on the film becomes minimum. However, when the exposing light source is ArF excimer laser light (wavelength is 193 nm), water is preferably used because of the ease of availability and ease of handling in addition to the above considerations. When water is used, a small proportion of an additive that decreases the surface tension of water and increases the surface activity may be added. Preferably, the additive cannot dissolve the resist film on the wafer and can neglect an influence on an optical coating at an under surface of a lens. The water used is preferably distilled water.

After the exposure, post exposure bake (PEB) is preferably carried out to promote the dissociation of the acid-dissociable group in the polymer by the acid generated from the radiation-sensitive acid generator with the exposure in the exposed part of the resist film. The difference of solubility into the developer between the exposed part and the non-exposed part is generated by the PEB. The temperature of PEB is typically from 50° C. to 180° C., and preferably from 80° C. to 130° C. The duration of PEB is typically from 5 seconds to 600 seconds, and preferably from 10 seconds to 300 seconds.

### [Developing Step]

In this step, the resist film exposed in the exposing step is developed. By this step, the predetermined resist pattern can be formed. After the development, the resist pattern is washed with a rinse solution such as water or alcohol, and the dried, in general.

Examples of the developer used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferred, and 2.38% by mass of aqueous TMAH solution is more preferred.

In the case of the development with organic solvent, examples of the solvent include an organic solvent, including a hydrocarbon-based solvent, an ether-based solvent, an ester-based solvent, a ketone-based solvent, and an alcohol-based solvent; and a solvent containing an organic solvent. Examples of the organic solvent include one, two or more solvents listed as the solvent for the radiation-sensitive composition. Among them, an ether-based solvent, an ester-based solvent or a ketone-based solvent is preferred. As the ether-based solvent, a glycol ether-based solvent is preferable, and ethylene glycol monomethyl ether and propylene glycol monomethyl ether are more preferable. The ester-based solvent is preferably an acetate ester-based solvent, and more preferably n-butyl acetate or amyl acetate. The ketone-based solvent is preferably a chain ketone, and more preferably 2-heptanone. The content of the organic solvent in the developer is preferably not less than 80% by mass, more preferably not less than 90% by mass, further preferably not less than 95% by mass, and particularly preferably not less than 99% by mass. Examples of the ingredient other than the organic solvent in the developer include water and silicone oil.

As described above, the developer may be either an alkaline developer or an organic solvent developer. The developer can be appropriately selected depending on whether the desired positive pattern or negative pattern is desired.

Examples of the developing method include a method of dipping the substrate in a tank filled with the developer for a given time (dip method); a method of developing by putting and leaving the developer on the surface of the substrate with the surface tension for a given time (paddle method); a method of spraying the developer on the surface of the substrate (spray method); and a method of injecting the developer while scanning an injection nozzle for the developer at a constant rate on the substrate rolling at a constant rate (dynamic dispense method).

### <Onium Salt Compound>

The onium salt compound according to still another embodiment is a compound represented by the following formula (1).

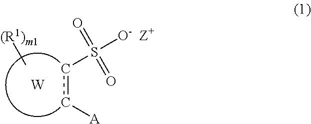

(In the formula (1),
W is a cyclic structure having 3 to 40 ring members formed together with two carbon atoms,
the following formula between carbon atoms represents a single bond or a double bond, [symbol]
A is a group represented by the following formula (A-1), a group represented by the following formula (A-2), a group represented by the following formula (A-3), a group represented by the following formula (A-4), a group represented by the following formula (A-5), a group represented by the following formula (A-6), or a group represented by the following formula (A-7),

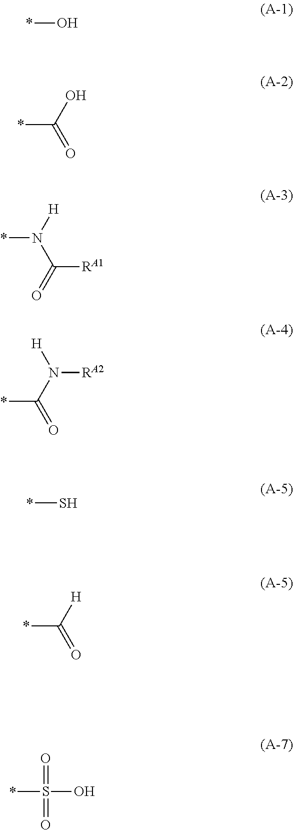

(In the formulas (A-3) and (A-4), R^{A1} and R^{A2} are each independently a monovalent hydrocarbon group having 1 to 20 carbon atoms, * is a bond with a carbon atom.)
R¹ is a monovalent organic group having 1 to 20 carbon atoms, a cyano group, a nitro group, a carboxy group, a hydroxy group, an amino group, a halogen atom, or a thiol group, when there are a plurality of R¹s, the plurality of R¹s are the same as or different from each other,
m₁ is an integer of 0 to 4, and
Z⁺ is a monovalent radiation-sensitive onium cation.)

As the onium salt compound represented by the formula (1a) according to the present embodiment, the onium salt compound (1) contained in the radiation-sensitive composition can be suitably used.

### EXAMPLES

Hereinafter, the present invention will be specifically described with reference to Examples, but the present invention is not limited to these Examples. Methods for measuring various physical property values are shown below.

### [Weight-Average Molecular Weight (Mw) and Number-Average Molecular Weight (Mn)]

The Mw and Mn of a polymer were measured under the conditions described above. A degree of dispersion (Mw/Mn) was calculated from results of the measured Mw and Mn.

[¹³C-NMR analysis]

¹³C-NMR analysis of the polymer was performed using a nuclear magnetic resonance apparatus (“JNM-Delta 400” manufactured by JEOL Ltd.).

### <Synthesis of Polymer>

Monomers used for synthesis of polymers in Examples and Comparative Examples are shown below. In the following synthesis examples, unless otherwise specified, “parts by mass” means a value taken when the total mass of the monomers used is 100 parts by mass, and “mol %” means a value taken when the total number of moles of the monomers used is 100 mol %.

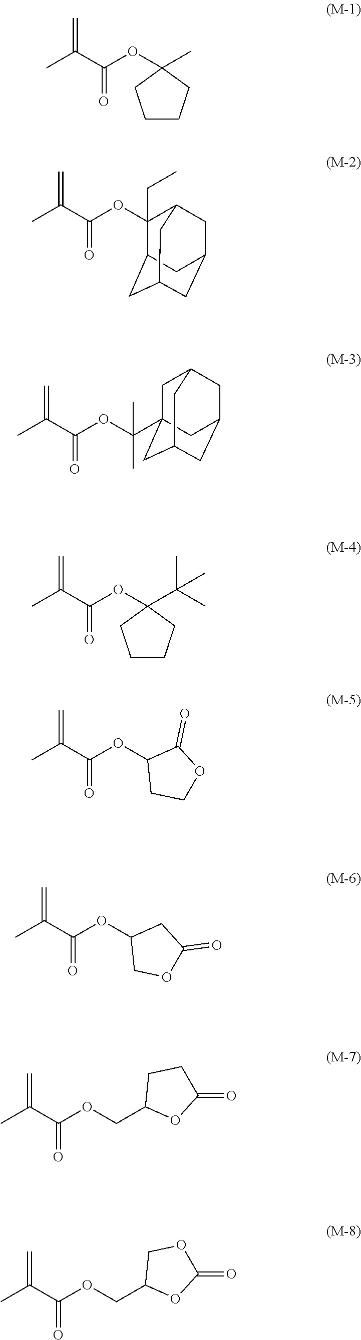

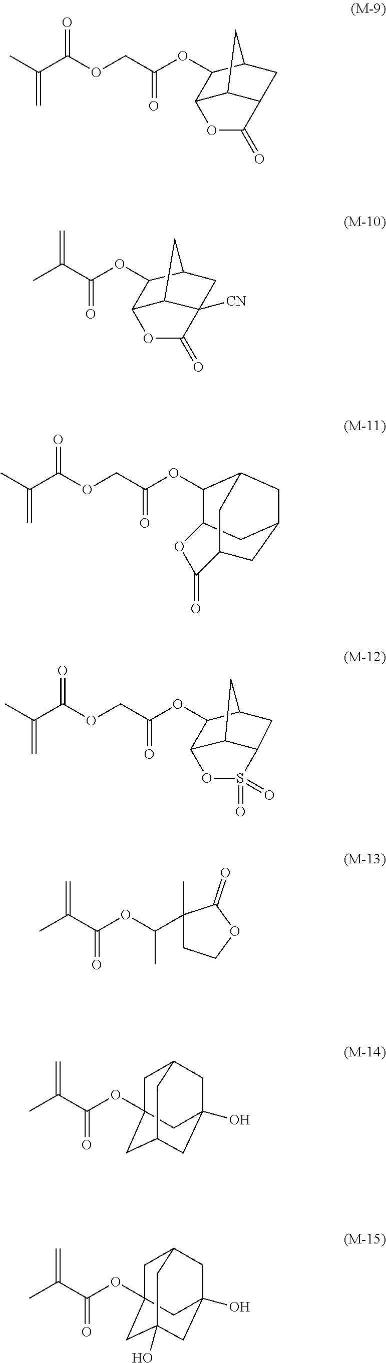

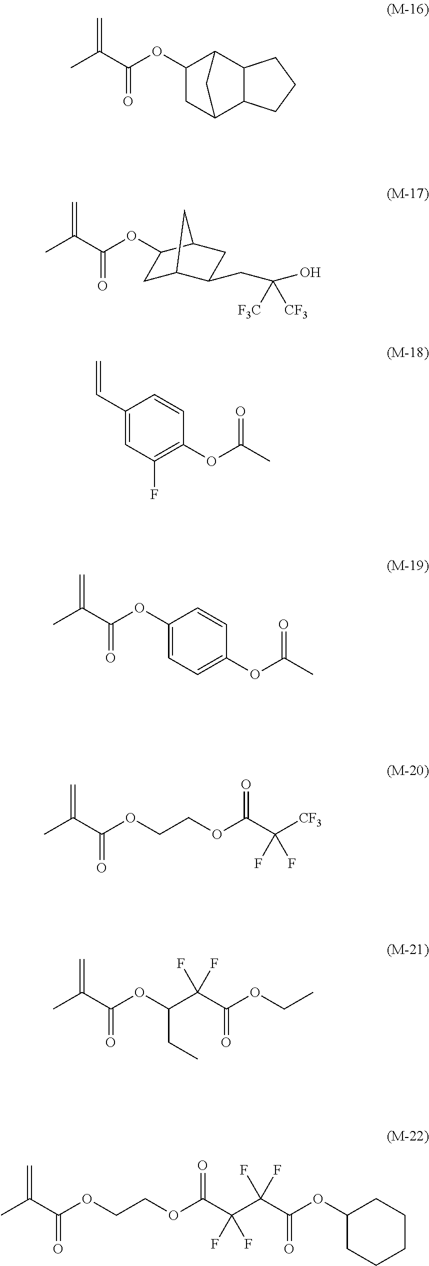

### Synthesis Example 1

(Synthesis of polymer (A-1))

A monomer (M-1), a monomer (M-2), a monomer (M-5), a monomer (M-10), and a monomer (M-14) were dissolved at a molar ratio of 40/10/20/20/10 (mol %) in 2-butanone (200 parts by mass), and AIBN (azobisisobutyronitrile) (5 mol based on 100 mol in total of the monomers used) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The polymerization solution cooled was poured into methanol (2,000 parts by mass), and a precipitated white powder was collected by filtration. The white powder separated by filtration was washed with methanol twice, then separated by filtration, and dried at 50° C. for 24 hours to obtain a white powdery polymer (A-1) (yield: 85%). The polymer (A-1) had an Mw of 7,100 and an Mw/Mn of 1.61. As a result of ³C-NMR analysis, the contents by percent of the structural units derived from (M-1), (M-2), (M-5), (M-10), and (M-14) were 40.3 mol %, 9.2 mol %, 20.5 mol %, 19.8 mol %, and 10.2 mol %, respectively.

### Synthesis Examples 2 to 11

### (Synthesis of Polymers (A-2) to (A-11))

Polymers (A-2) to (A-11) were synthesized in the same manner as in Synthesis Example 1 except that monomers of types and blending ratios shown in the following Table 1 were used. The content by percent (mol %) and physical property values (Mw and Mw/Mn) of each of the structural units of the resulting polymers are also shown in Table 1. In Table 1, “-” indicates that the corresponding monomer was not used (the same applies to Tables below).

[symbol]

TABLE 1

| | | Monomer that affords structural | | | Monomer that affords structural | | | Monomer that affords structural | | | | |
|---|---|---|---|---|---|---|---|---|---|---|---|---|
| | | unit (I) | | | unit (II) | | | unit (III) or (VII) | | | | |
| | | | | Content ratio | | | Content ratio | | | Content ratio | | |
| | | | Blending | of structural | | Blending | of structural | | Blending | of structural | | |
| | [A] | | ratio | unit | | ratio | unit | | ratio | unit | | Mw/ |
| | Polymer | Type | (mol %) | (mol %) | Type | (mol %) | (mol %) | Type | (mol %) | (mol %) | Mw | Mn |
| Synthesis | A-1 | M-1 | 40 | 40.3 | M-5 | 20 | 20.5 | M-14 | 10 | 10.2 | 7100 | 1.61 |
| Example 1 | | M-2 | 10 | 9.2 | M-10 | 20 | 19.8 | | | | | |
| Synthesis | A-2 | M-1 | 30 | 30.2 | M-9 | 50 | 50.6 | — | — | — | 7700 | 1.51 |
| Example 2 | | M-2 | 20 | 19.2 | | | | | | | | |
| Synthesis | A-3 | M-1 | 30 | 31.0 | M-11 | 50 | 49.4 | — | — | — | 7200 | 1.59 |
| Example 3 | | M-3 | 20 | 19.6 | | | | | | | | |
| Synthesis | A-4 | M-1 | 40 | 40.5 | M-12 | 50 | 49.2 | — | — | — | 6800 | 1.61 |
| Example 4 | | M-3 | 10 | 10.3 | | | | | | | | |
| Synthesis | A-5 | M-1 | 40 | 40.6 | M-13 | 50 | 51.3 | — | — | — | 6900 | 1.44 |
| Example 5 | | M-4 | 10 | 8.1 | | | | | | | | |
| Synthesis | A-6 | M-1 | 40 | 40.9 | M-6 | 20 | 20.5 | M-16 | 10 | 10.0 | 7500 | 1.51 |
| Example 6 | | M-4 | 10 | 8.3 | M-9 | 20 | 20.3 | | | | | |
| Synthesis | A-7 | M-1 | 50 | 50.2 | M-10 | 30 | 29.6 | M-14 | 20 | 20.2 | 7200 | 1.55 |
| Example 7 | | | | | | | | | | | | |
| Synthesis | A-8 | M-1 | 40 | 40.0 | M-7 | 20 | 20.5 | M-15 | 10 | 9.2 | 7100 | 1.62 |
| Example 8 | | M-3 | 10 | 10.1 | M-11 | 20 | 20.2 | | | | | |
| Synthesis | A-9 | M-1 | 50 | 50.3 | M-8 | 50 | 49.7 | — | — | — | 7000 | 1.51 |
| Example 9 | | | | | | | | | | | | |
| Synthesis | A-10 | M-1 | 40 | 40.2 | M-9 | 60 | 59.8 | — | — | — | 6700 | 1.50 |
| Example 10 | | | | | | | | | | | | |
| Synthesis | A-11 | M-2 | 40 | 39.4 | M-10 | 60 | 60.6 | — | — | — | 7500 | 1.49 |
| Example 11 | | | | | | | | | | | | |

### (Synthesis of Polymer (A-12))

Monomers (M-1) and (M-18) were dissolved at a molar ratio of 50/50 (mol %) in 1-methoxy-2 propanol (200 parts by mass), and AIBN (5 mol %) was added thereto as an initiator to prepare a monomer solution. 1-methoxy-2-propanol (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The cooled polymerization solution was poured into hexane (2,000 parts by mass), and a precipitated white powder was collected by filtration. The white powder separated by filtration was washed with hexane twice, then separated by filtration, and dissolved in 1-methoxy-2-propanol (300 parts by mass). Next, methanol (500 parts by mass), triethylamine (50 parts by mass) and ultrapure water (10 parts by mass) were added, and a hydrolysis reaction was performed at 70° C. for 6 hours with stirring. After the completion of the reaction, the remaining solvent was distilled off. The resulting solid was dissolved in acetone (100 parts by mass), and the solution was added dropwise to water (500 parts by mass) to solidify a polymer. The resulting solid was separated by filtration, and dried at 50° C. for 13 hours to obtain a white powdery polymer (A-12) (yield: 81%). The polymer (A-12) had an Mw of 5,500 and an Mw/Mn of 1.61. As a result of ¹³C-NMR analysis, the contents by percent of the structural units derived from (M-1) and (M-18) were respectively 50.2 mol % and 49.8 mol %.

### Synthesis Examples 13 to 15

### (Synthesis of Polymers (A-13) to (A-15))

Polymers (A-13) to (A-15) were synthesized in the same manner as in Synthesis Example 12 except that monomers of types and blending ratios shown in the following Table 2 were used. For the monomer providing the structural unit (IV), in the polymer, the disappearance of the peak of the carbonyl group of the acetyl group was confirmed by measurement of ¹³C-NMR, and substantially all the alkali-dissociable groups were hydrolyzed to the phenolic hydroxyl group. The content by percent (mol %) of each of the structural units and the physical property values (Mw and Mw/Mn) of the polymers obtained are shown together in Table 2.

TABLE 2

| | | Monomer that affords structural | | | Monomer that affords structural | | | Monomer that affords structural | | | | |
|---|---|---|---|---|---|---|---|---|---|---|---|---|
| | | unit (I) | | | unit (III) | | | unit (IV) | | | | |
| | | | | Content ratio | | | Content ratio | | | Content ratio | | |
| | | | Blending | of structural | | Blending | of structural | | Blending | of structural | | |
| | [A] | | ratio | unit | | ratio | unit | | ratio | unit | | Mw/ |
| | Polymer | Type | (mol %) | (mol %) | Type | (mol %) | (mol %) | Type | (mol %) | (mol %) | Mw | Mn |
| Synthesis | A-12 | M-1 | 50 | 50.2 | — | — | — | M-18 | 50 | 49.8 | 5500 | 1.62 |
| Example 12 | | | | | | | | | | | | |
| Synthesis | A-13 | M-3 | 50 | 46.6 | M-14 | 10 | 11.1 | M-19 | 40 | 42.3 | 5600 | 1.55 |
| Example 13 | | | | | | | | | | | | |
| Synthesis | A-14 | M-2 | 50 | 48.1 | M-17 | 20 | 21.3 | M-18 | 30 | 30.6 | 5100 | 1.59 |
| Example 14 | | | | | | | | | | | | |
| Synthesis | A-15 | M-4 | 55 | 53.2 | M-17 | 15 | 15.2 | M-19 | 30 | 31.6 | 6100 | 1.50 |
| Example 15 | | | | | | | | | | | | |

### Synthesis Example 16

### [Synthesis of High Fluorine-Containing Polymer (F-1)]

Monomers (M-1) and (M-20) were dissolved at a molar ratio of 20/80 (mol %) in 2-butanone (200 parts by mass), and AIBN (4 mol %) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The solvent was replaced with acetonitrile (400 parts by mass). Hexane (100 parts by mass) was then added, followed by stirring, and an acetonitrile layer was collected. The operation was repeated three times. By replacing the solvent with propylene glycol monomethyl ether acetate, a solution of a high fluorine-containing polymer (F-1) was obtained (yield: 75%). The high fluorine-containing polymer (F-1) had an Mw of 6,200 and an Mw/Mn of 1.77. As a result of ¹³C-NMR analysis, the contents by percent of the structural units derived from (M-1) and (M-20) were respectively 19.5 mol % and 80.5 mol %.

### Synthesis Examples 17 to 20

### (Synthesis of High Fluorine-Containing Polymers (F-2) to (F-5))

High fluorine-containing polymers (F-2) to (F-5) were synthesized in the same manner as in Synthesis Example 16 except that monomers of types and blending ratios shown in Table 3 were used. The content by percent (mol %) and physical property values (Mw and Mw/Mn) of each of the structural units of the resulting high fluorine-containing polymers are also shown in Table 3.

TABLE 3

| | | Monomer that affords | | | Monomer that affords | | |
|---|---|---|---|---|---|---|---|
| | | structural unit (VI) | | | structural unit (I) | | |
| | [F] | | | Content | | | Content |
| | High | | | ratio of | | | ratio of |
| | fluorine- | | Blending | structural | | Blending | structural |
| | content | | ratio | unit | | ratio | unit |
| | polymer | Type | (mol %) | (mol %) | Type | (mol %) | (mol %) |
| Synthesis | F-1 | M-20 | 80 | 80.5 | M-1 | 20 | 19.5 |
| Example | | | | | | | |
| 16 | | | | | | | |
| Synthesis | F-2 | M-21 | 80 | 80.9 | M-4 | 20 | 19.1 |
| Example | | | | | | | |
| 17 | | | | | | | |
| Synthesis | F-3 | M-22 | 60 | 62.3 | — | — | — |
| Example | | | | | | | |
| 18 | | | | | | | |
| Synthesis | F-4 | M-22 | 60 | 60.2 | M-2 | 20 | 19.4 |
| Example | | | | | | | |
| 19 | | | | | | | |
| Synthesis | F-5 | M-20 | 60 | 60.0 | M-3 | 10 | 10.1 |
| Example | | | | | | | |
| 20 | | | | | | | |

| | Monomer that affords | | | Monomer that affords | | | | |
|---|---|---|---|---|---|---|---|---|
| | structural unit (III) | | | structural unit (VII) | | | | |
| | | | Content | | | Content | | |
| | | | ratio of | | | ratio of | | |
| | | Blending | structural | | Blending | structural | | |
| | | ratio | unit | | ratio | unit | | Mw/ |
| | Type | (mol %) | (mol %) | Type | (mol %) | (mol %) | Mw | Mn |
| Synthesis | — | — | — | — | — | — | 6200 | 1.77 |
| Example | | | | | | | | |
| 16 | | | | | | | | |
| Synthesis | — | — | — | — | — | — | 7100 | 1.82 |
| Example | | | | | | | | |
| 17 | | | | | | | | |
| Synthesis | — | — | — | M-16 | 40 | 37.7 | 6900 | 1.91 |
| Example | | | | | | | | |
| 18 | | | | | | | | |
| Synthesis | M-14 | 20 | 20.4 | — | — | — | 7300 | 1.88 |
| Example | | | | | | | | |
| 19 | | | | | | | | |
| Synthesis | M-17 | 30 | 29.9 | — | — | — | 6700 | 1.87 |
| Example | | | | | | | | |
| 20 | | | | | | | | |

### <[B] Synthesis of Onium Salt Compound (1)>

Compounds (B-1) to (B-10) as the onium salt compound (1) which is a radiation-sensitive acid generator were synthesized as follows.

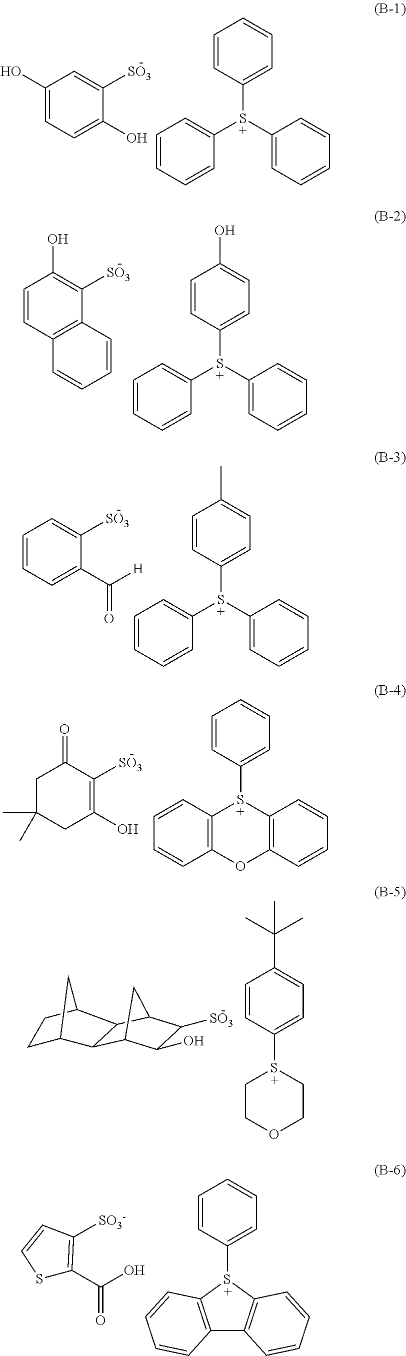

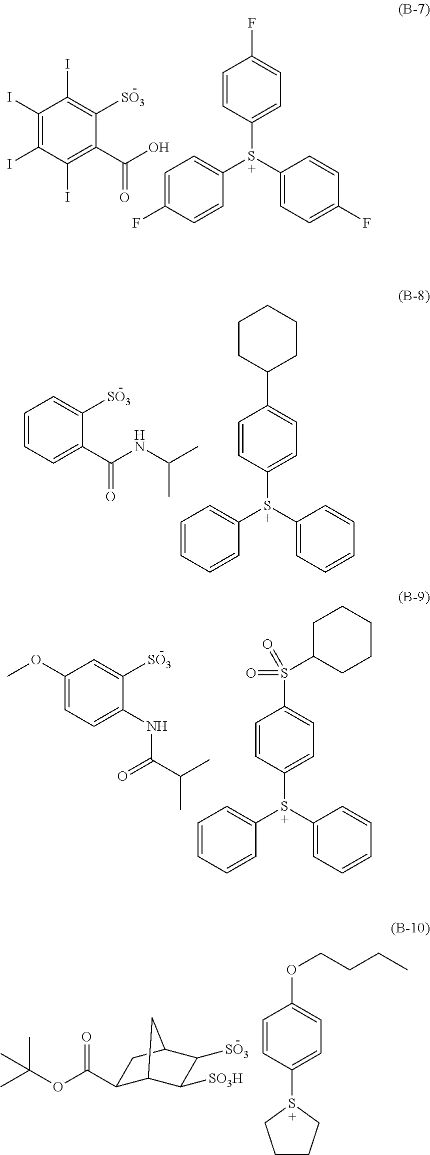

### Example B1

### (Synthesis of Compound (B-1))

A compound (B-1) was synthesized according to the following synthesis scheme.

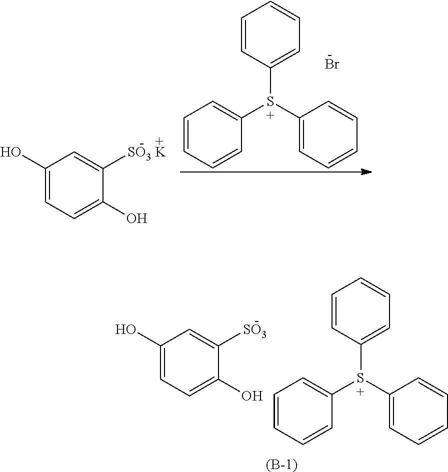

To a reaction vessel, 20.0 mmol of potassium hydroquinone sulfonate and 20.0 mmol of triphenylsulfonium bromide were added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-1) represented by the formula (B-1) in a good yield.

### Examples B2 to B3

### (Synthesis of Compounds (B-2) to (B-3))

Onium salt compounds (1) represented by the formulas (B-2) to (B-3) were synthesized in the same manner as in Example B1 except that the raw materials and the precursor were appropriately changed.

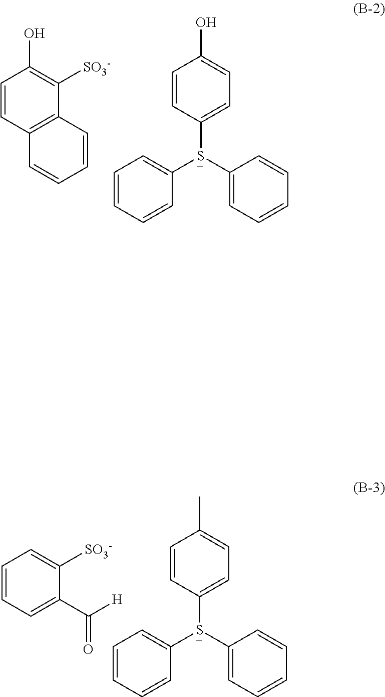

### Example B4

### (Synthesis of Compound (B-4))

A compound (B-4) was synthesized according to the following synthesis scheme.

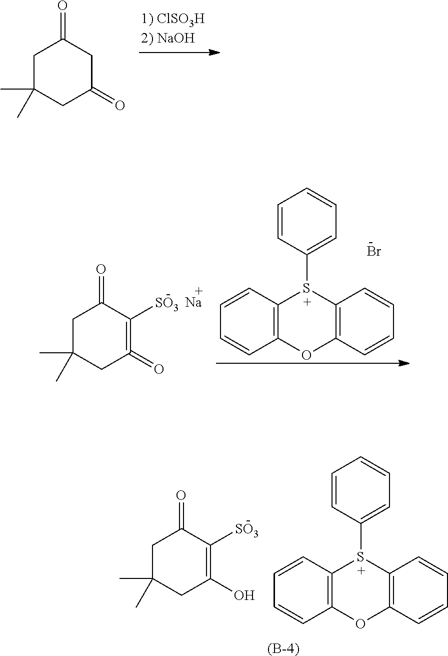

To a reaction vessel, 20.0 mmol of dimedone, 30.0 mmol of chlorosulfonic acid, and 1,4 dioxane were added to form a 0.5 M solution. After reaction at 70° C. for 6 hours, an aqueous sodium hydroxide solution was added to stop the reaction. Methylene chloride was added for extraction, and then the organic layer was separated. The resulting organic layer was dried over sodium sulfate, and then the solvent was distilled off to afford a sodium sulfonate salt in a good yield.

To the sodium sulfonate salt, 20.0 mmol of sulfonium bromide was added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-4) represented by the formula (B-4) in a good yield.

### Example B5

(Synthesis of compound (B-5))

A compound (B-5) was synthesized according to the following synthesis scheme.

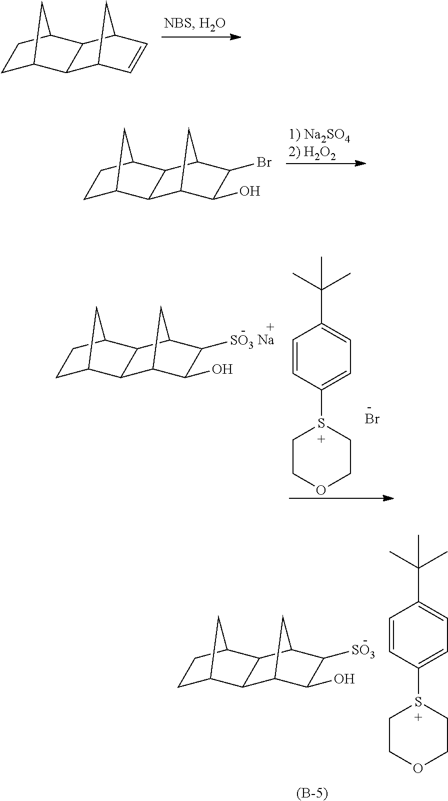

To a reaction vessel, 20.0 mmol of tetracyclododecene and 30.0 mmol of N-bromosuccinimide were added, and a mixed liquid of water and acetone (1:5 (mass ratio)) was added to form a 0.5 M solution. After reaction at 0° C. for 1 hour, an aqueous sodium hydrogen carbonate solution was added to stop the reaction. After the acetone was distilled off, methylene chloride was added for extraction, and then the organic layer was separated. After drying the resulting organic layer over sodium sulfate, the solvent was distilled off, and purification was performed by column chromatography, affording a brominated product in a good yield.

A mixed liquid of acetonitrile and water (1:1 (mass ratio)) was added to the brominated product to form a 1 M solution. Then, 40.0 mmol of sodium dithionite and 60.0 mmol of sodium hydrogen carbonate were added, followed by reaction at 70° C. for 6 hours. After extraction with acetonitrile and distillation of the solvent, a mixed liquid of acetonitrile and water (3:1 (mass ratio)) was added to form a 0.5 M solution. To the solution were added 60.0 mmol of hydrogen peroxide and 2.00 mmol of sodium tungstate, followed by heating and stirring at 50° C. for 12 hours. The mixture was extracted with acetonitrile, and the solvent was distilled off, affording a sodium sulfonate salt.

To the sodium sulfonate salt, 20.0 mmol of sulfonium bromide was added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-5) represented by the formula (B-5) in a good yield.

### Example B6

(Synthesis of compound (B-6))

A compound (B-6) was synthesized according to the following synthesis scheme.

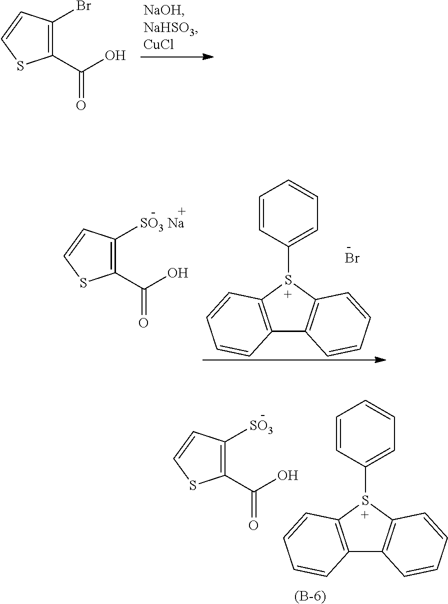

To a reaction vessel, 20.0 mmol of 3-bromothiophene-2-carboxylic acid, 24.0 mmol of sodium bisulfite, and 2.0 mmol of copper chloride were added, and an aqueous solution of sodium hydroxide was added to adjust the pH to 7.5-7.7. After reaction at 100° C. for 3 hours, the insoluble matter was removed by filtration. The collected filtrate was cooled to 0° C. and allowed to stand for 12 hours, and then the precipitated crystal was filtered off. The filtered-off crystal was washed with acetone to afford a sodium sulfonate salt in a good yield.

To the sodium sulfonate salt, 20.0 mmol of sulfonium bromide was added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-6) represented by the formula (B-6) in a good yield.

### Example B7

### (Synthesis of Compound (B-7))

A compound (B-7) was synthesized according to the following synthesis scheme.

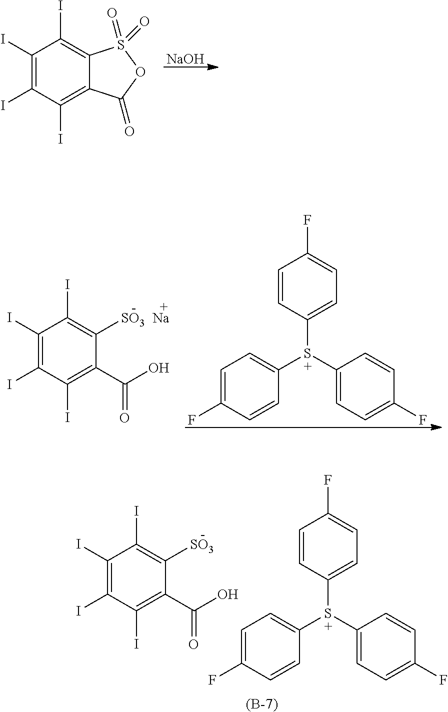

To a reaction vessel, 20.0 mmol of tetraiodo-2-sulfobenzoic acid anhydride and an aqueous sodium hydroxide solution were added to form a 0.5 M solution. After reaction at 70° C. for 6 hours, methylene chloride was added for extraction, and then the organic layer was separated. The resulting organic layer was dried over sodium sulfate, and then the solvent was distilled off to afford a sodium sulfonate salt in a good yield.

To the sodium sulfonate salt, 20.0 mmol of sulfonium bromide was added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-7) represented by the formula (B-7) in a good yield.

### Example B8

### (Synthesis of Compound (B-8))

A compound (B-8) was synthesized according to the following synthesis scheme.

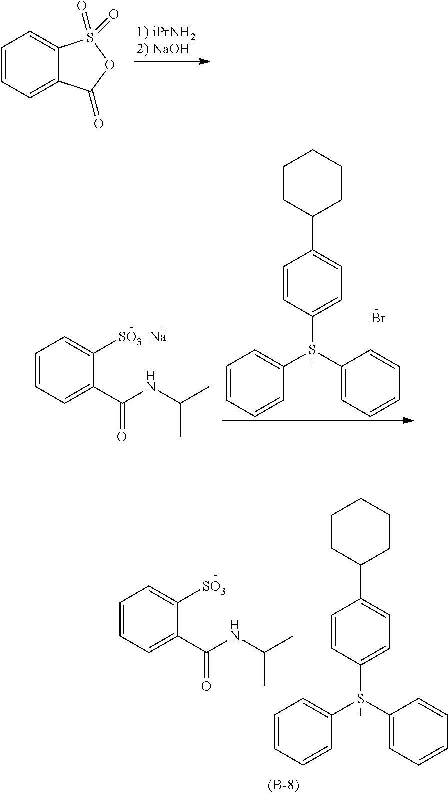

To a reaction vessel, 20.0 mmol of 2-sulfobenzoic acid anhydride, 30.0 mmol of isopropylamine, and tetrahydrofuran were added to form a 0.5 M solution. After reaction at 70° C. for 6 hours, an aqueous sodium hydroxide solution was added to stop the reaction. Methylene chloride was added for extraction, and then the organic layer was separated. The resulting organic layer was dried over sodium sulfate, and then the solvent was distilled off to afford a sodium sulfonate salt in a good yield.

To the sodium sulfonate salt, 20.0 mmol of sulfonium bromide was added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-8) represented by the formula (B-8) in a good yield.

### Example B9

### (Synthesis of Compound (B-9))

A compound (B-9) was synthesized according to the following synthesis scheme.

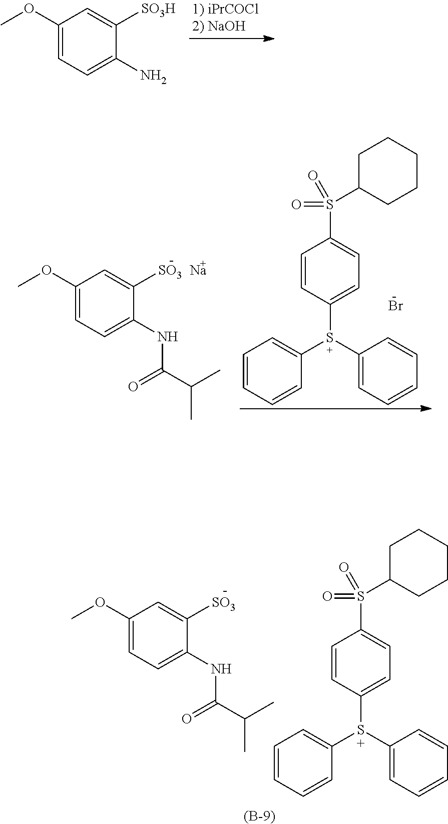

To a reaction vessel, 20.0 mmol of 2-amino-5-methoxybenzenesulfonic acid, 30.0 mmol of isobutyl chloride, and tetrahydrofuran were added to form a 0.5 M solution. After reaction at 70° C. for 6 hours, an aqueous sodium hydroxide solution was added to stop the reaction. Methylene chloride was added for extraction, and then the organic layer was separated. The resulting organic layer was dried over sodium sulfate, and then the solvent was distilled off to afford a sodium sulfonate salt in a good yield.

To the sodium sulfonate salt, 20.0 mmol of sulfonium bromide was added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-9) represented by the formula (B-9) in a good yield.

### Example B10

### (Synthesis of Compound (B-10))

A compound (B-10) was synthesized according to the following synthesis scheme.

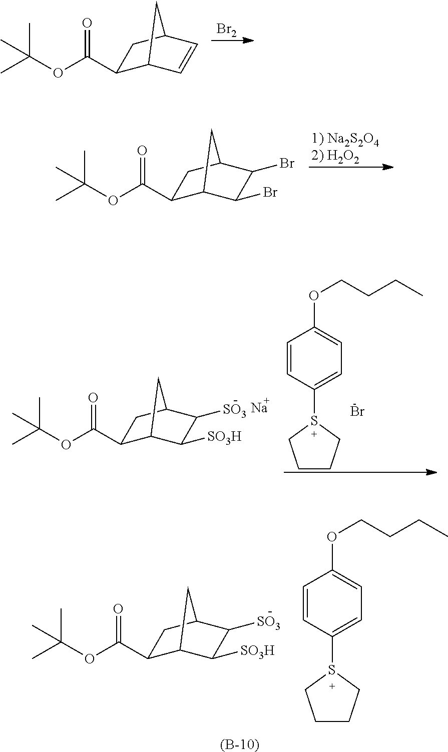

To a reaction vessel, 20.0 mmol of tert-butyl 5-norbornene-2-carboxylate, 30.0 mmol of bromine, and methylene chloride were added to form a 0.5 M solution. After reaction at room temperature for 6 hours, water was added to stop the reaction. Methylene chloride was added for extraction, and then the organic layer was separated. The resulting organic layer was dried over sodium sulfate, and then the solvent was distilled off to afford a brominated product in a good yield.

A mixed liquid of acetonitrile and water (1:1 (mass ratio)) was added to the brominated product to form a 1 M solution. Then, 60.0 mmol of sodium dithionite and 80.0 mmol of sodium hydrogen carbonate were added, followed by reaction at 70° C. for 6 hours. After extraction with acetonitrile and distillation of the solvent, a mixed liquid of acetonitrile and water (3:1 (mass ratio)) was added to form a 0.5 M solution. To the solution were added 60.0 mmol of hydrogen peroxide and 2.00 mmol of sodium tungstate, followed by heating and stirring at 50° C. for 12 hours. The mixture was extracted with acetonitrile, and the solvent was distilled off, affording a sodium sulfonate salt.

To the sodium sulfonate salt, 20.0 mmol of sulfonium bromide was added, and a mixed liquid of water and methylene chloride (1:3 (mass ratio)) was added to form a 0.5 M solution. After vigorous stirring at room temperature for 3 hours, methylene chloride was added for extraction, and then the organic layer was separated. The resulting organic layer was dried over sodium sulfate, the solvent was then distilled off, and purification was performed by column chromatography, affording a compound (B-10) represented by the formula (B-10) in a good yield.

The following compounds were used as components other than the synthesized components.

[Radiation-Sensitive Acid Generator Other than Compounds (B-1) to (B-10) as Onium Salt Compound (1)]

b-1 to b-11: Compounds represented by formulae (b-1) to (b-11) (hereinafter, the compounds represented by formulae (b-1) to (b-11) may be described as “compound (b-1)” to “compound (b-11)”, respectively).

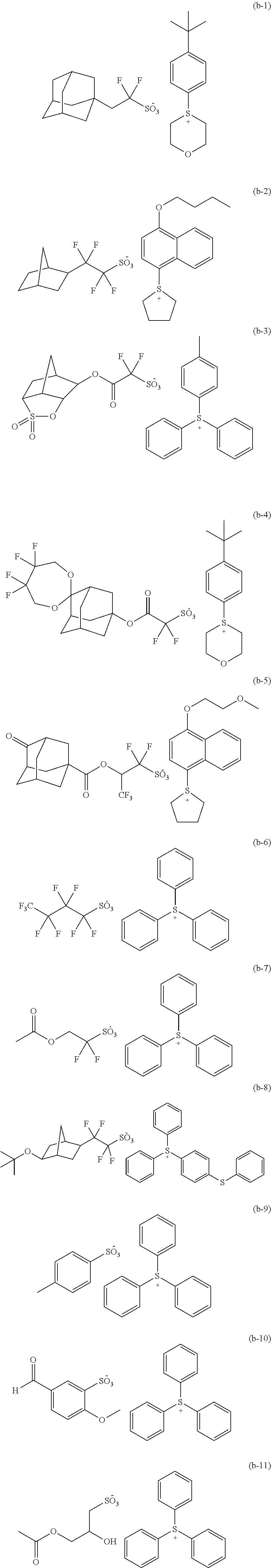

### [Acid Diffusion Controlling Agent [D]]

D-1 to D-7: Compounds represented by the formulas (D-1) to (D-7)

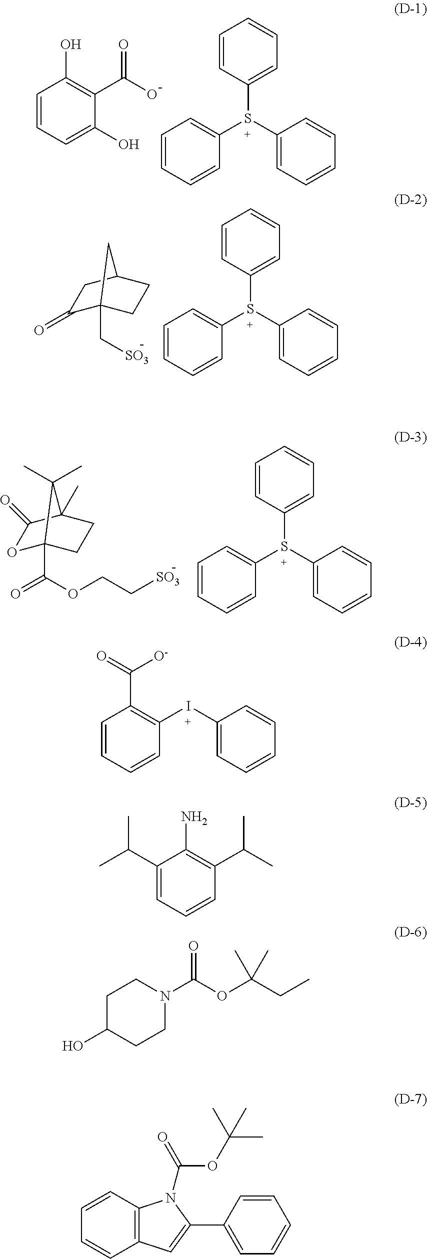

### [Solvent [E]]

E-1: propylene glycol monomethyl ether acetate
E-2: cyclohexanone
E-3: γ-butyrolactone
E-4: ethyl lactate

### [Preparation of Positive Radiation-Sensitive Composition for ArF Immersion Exposure]

### Example 1

100 parts by mass of (A-1) as a polymer [A], 10.0 parts by mass of (B-1) as an onium salt compound (1) [B], 4.0 parts by mass of (D-1) as an acid diffusion controlling agent [D], 5.0 parts by mass (solid content) of (F-1) as a high fluorine-content polymer [F], and 3,400 parts by mass of a mixed solvent of (E-1)/(E-2)/(E-3) as a solvent [E] were mixed, and the mixture was filtered through a membrane filter having a pore size of 0.2 μm to prepare a radiation-sensitive composition (J-1).

### Examples 2 to 38 and Comparative Examples 1 to 3

Radiation-sensitive compositions (J-2) to (J-38) and (CJ-1) to (CJ-3) were prepared in the same manner as in Example 1 except that the components of the types and the contents shown in Table 4 were used.

TABLE 4

| | | | | | | | | [F] High | | | |
|---|---|---|---|---|---|---|---|---|---|---|---|
| | | | | [B] Onium salt | | [D] Acid diffusion | | fluorine-content | | | |
| | | [A] Polymer | | compound (1) | | controlling agent | | polymer | | [E] Solvent | |
| | Radiation- | | Content | | Content | | Content | | Content | | Content |
| | sensitive | | (parts by | | (parts by | | (parts by | | (parts by | | (parts by |
| | composition | Type | mass) | Type | mass) | Type | mass) | Type | mass) | Type | mass) |
| Example 1 | J-1 | A-1 | 100 | B-1 | 10.0 | D-1 | 4.0 | F-1 | 5.0 | E-1/E-2/E-3 | 2240/960/200 |
| Example 2 | J-2 | A-2 | 100 | B-1 | 10.0 | D-1 | 4.0 | F-1 | 5.0 | E-1/E-2/E-3 | 2240/960/200 |
| Example 3 | J-3 | A-3 | 100 | B-1 | 10.0 | D-1 | 4.0 | F-1 | 5.0 | E-1/E-2/E-3 | 2240/960/200 |
| Example 4 | J-4 | A-4 | 100 | B-1 | 10.0 | D-1 | 4.0 | F-1 | 5.0 | E-1/E-2/E-3 | 2240/960/200 |
| Example 5 | J-5 | A-5 | 100 | B-1 | 10.0 | D-1 | 4.0 | F-1 | 5.0 | E-1/E-2/E-3 | 2240/960/200 |
| Example 6 | J-6 | A-6 | 100 | B-1 | 10.0 | D-1 | 4.0 | F-1 | 5.0 | E-1/E-2/E-3 | 2240/960/200 |
| Example 7 | J-7 | A-7 | 100 | B-1 | 10.0 | D-1 | 4.0 | F-1 | 5.0 | E-1/E-2/E-3 | 2240/960/200 |
| Example 8 | J-8 | A-8 | 100 | B-1 | 10.0 | D-1 | 4.0 | F-1 | 5.0 | E-1/E-2/E-3 | 2240/960/200 |
| Example 9 | J-9 | A-9 | 100 | B-1 | 10.0 | D-1 | 4.0 | F-1 | 5.0 | E-1/E-2/E-3 | 2240/960/200 |
| Example 10 | J-10 | A-10 | 100 | B-1 | 10.0 | D-1 | 4.0 | F-1 | 5.0 | E-1/E-2/E-3 | 2240/960/200 |
| Example 11 | J-11 | A-11 | 100 | B-1 | 10.0 | D-1 | 4.0 | F-1 | 5.0 | E-1/E-2/E-3 | 2240/960/200 |
| Example 12 | J-12 | A-1 | 100 | B-1 | 10.0 | D-2 | 4.0 | F-1 | 5.0 | E-1/E-2/E-3 | 2240/960/200 |
| Example 13 | J-13 | A-1 | 100 | B-1 | 10.0 | D-3 | 4.0 | F-1 | 5.0 | E-1/E-2/E-3 | 2240/960/200 |
| Example 14 | J-14 | A-1 | 100 | B-1 | 10.0 | D-4 | 4.0 | F-1 | 5.0 | E-1/E-2/E-3 | 2240/960/200 |
| Example 15 | J-15 | A-1 | 100 | B-1 | 10.0 | D-5 | 4.0 | F-1 | 5.0 | E-1/E-2/E-3 | 2240/960/200 |
| Example 16 | J-16 | A-1 | 100 | B-1 | 10.0 | D-6 | 4.0 | F-1 | 5.0 | E-1/E-2/E-3 | 2240/960/200 |
| Example 17 | J-17 | A-1 | 100 | B-1 | 10.0 | D-7 | 4.0 | F-1 | 5.0 | E-1/E-2/E-3 | 2240/960/200 |
| Example 18 | J-18 | A-1 | 100 | B-2 | 5.0 | D-1 | 4.0 | F-1 | 5.0 | E-1/E-2/E-3 | 2240/960/200 |
| Example 19 | J-19 | A-1 | 100 | B-3 | 10.0 | D-1 | 4.0 | F-1 | 5.0 | E-1/E-2/E-3 | 2240/960/200 |
| Example 20 | J-20 | A-1 | 100 | B-4 | 10.0 | D-1 | 4.0 | F-1 | 5.0 | E-1/E-2/E-3 | 2240/960/200 |
| Example 21 | J-21 | A-1 | 100 | B-5 | 10.0 | D-1 | 4.0 | F-1 | 5.0 | E-1/E-2/E-3 | 2240/960/200 |
| Example 22 | J-22 | A-1 | 100 | B-6 | 10.0 | D-1 | 4.0 | F-1 | 5.0 | E-1/E-2/E-3 | 2240/960/200 |
| Example 23 | J-23 | A-1 | 100 | B-7 | 10.0 | D-1 | 4.0 | F-1 | 5.0 | E-1/E-2/E-3 | 2240/960/200 |
| Example 24 | J-24 | A-1 | 100 | B-8 | 10.0 | D-1 | 4.0 | F-1 | 5.0 | E-1/E-2/E-3 | 2240/960/200 |
| Example 25 | J-25 | A-1 | 100 | B-9 | 10.0 | D-1 | 4.0 | F-1 | 5.0 | E-1/E-2/E-3 | 2240/960/200 |
| Example 26 | J-26 | A-1 | 100 | B-10 | 10.0 | D-1 | 4.0 | F-1 | 5.0 | E-1/E-2/E-3 | 2240/960/200 |
| Example 27 | J-27 | A-1 | 100 | B-1/b-1 | 5.0/5.0 | D-1 | 4.0 | F-1 | 5.0 | E-1/E-2/E-3 | 2240/960/200 |
| Example 28 | J-28 | A-1 | 100 | B-1/b-2 | 5.0/5.0 | D-1 | 4.0 | F-1 | 5.0 | E-1/E-2/E-3 | 2240/960/200 |
| Example 29 | J-29 | A-1 | 100 | B-1/b-3 | 5.0/5.0 | D-1 | 4.0 | F-1 | 5.0 | E-1/E-2/E-3 | 2240/960/200 |
| Example 30 | J-30 | A-1 | 100 | B-1/b-4 | 5.0/5.0 | D-1 | 4.0 | F-1 | 5.0 | E-1/E-2/E-3 | 2240/960/200 |
| Example 31 | J-31 | A-1 | 100 | B-1/b-5 | 5.0/5.0 | D-1 | 4.0 | F-1 | 5.0 | E-1/E-2/E-3 | 2240/960/200 |
| Example 32 | J-32 | A-1 | 100 | B-1/b-6 | 5.0/5.0 | D-1 | 4.0 | F-1 | 5.0 | E-1/E-2/E-3 | 2240/960/200 |
| Example 33 | J-33 | A-1 | 100 | B-1/b-7 | 5.0/5.0 | D-1 | 4.0 | F-1 | 5.0 | E-1/E-2/E-3 | 2240/960/200 |
| Example 34 | J-34 | A-1 | 100 | B-1/b-8 | 5.0/5.0 | D-1 | 4.0 | F-1 | 5.0 | E-1/E-2/E-3 | 2240/960/200 |
| Example 35 | J-35 | A-1 | 100 | B-5/B-10 | 5.0/5.0 | D-1 | 4.0 | F-1 | 5.0 | E-1/E-2/E-3 | 2240/960/200 |
| Example 36 | J-36 | A-1 | 100 | B-1 | 10.0 | D-1 | 4.0 | F-2 | 5.0 | E-1/E-2/E-3 | 2240/960/200 |
| Example 37 | J-37 | A-1 | 100 | B-1 | 10.0 | D-1 | 4.0 | F-3 | 5.0 | E-1/E-2/E-3 | 2240/960/200 |
| Example 38 | J-38 | A-1 | 100 | B-1 | 10.0 | D-1 | 4.0 | F-4 | 5.0 | E-1/E-2/E-3 | 2240/960/200 |
| Comparative | CJ-1 | A-1 | 100 | b-9 | 10.0 | D-1 | 4.0 | F-1 | 5.0 | E-1/E-2/E-3 | 2240/960/200 |
| Example 1 | | | | | | | | | | | |
| Comparative | CJ-2 | A-1 | 100 | b-10 | 10.0 | D-1 | 4.0 | F-1 | 5.0 | E-1/E-2/E-3 | 2240/960/200 |
| Example 2 | | | | | | | | | | | |
| Comparative | CJ-3 | A-1 | 100 | b-11 | 10.0 | D-1 | 4.0 | F-1 | 5.0 | E-1/E-2/E-3 | 2240/960/200 |
| Example 3 | | | | | | | | | | | |

### <Formation of Resist Pattern Using Positive Radiation-Sensitive Composition for ArF Immersion Exposure>

Onto the surface of a 12-inch silicon wafer, an underlayer antireflection film forming composition (“ARC66” manufactured by Brewer Science Incorporated) was applied with use of a spin coater (“CLEAN TRACK ACT12” manufactured by Tokyo Electron Limited). The wafer was then heated at 205° C. for 60 seconds to form an underlayer antireflection film having an average thickness of 100 nm. The positive radiation-sensitive composition for ArF immersion exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 90 nm. Next, the resist film was exposed through a 60 nm line-and-space mask pattern using an ArF excimer laser immersion exposure apparatus (“TWINSCAN XT-1900i” manufactured by ASML) with NA of 1.35 under an optical condition of Dipole (σ=0.9/0.7). After the exposure, PEB (post exposure baking) was performed at 100° C. for 60 seconds. Thereafter, the resist film was developed with an alkali with use of a 2.38% by mass aqueous TMAH solution as an alkaline developer, followed by washing with water and further drying to form a positive resist pattern (60 nm line-and-space pattern).

### <Evaluation>

The resist patterns formed using the positive radiation-sensitive composition for ArF immersion exposure were evaluated on sensitivity, LWR performance, and storage stability according to the following methods. The results are shown in the following Table 5. A scanning electron microscope (“CG-5000” manufactured by Hitachi High-Tech Corporation) was used for measuring the length of the resist pattern.

### [Sensitivity]

An exposure dose at which a 60 nm line-and-space pattern was formed in the aforementioned resist pattern formation using each of the positive radiation-sensitive compositions for ArF immersion exposure was defined as an optimum exposure dose, and this optimum exposure dose was defined as sensitivity (mJ/cm²). In a case of being 30 mJ/cm² or less, the sensitivity was evaluated as “good”, and in a case of being more than 30 mJ/cm², the sensitivity was evaluated as “poor”.

### [LWR Performance]

A 60 nm line-and-space resist pattern was formed by irradiation with the optimum exposure dose obtained in the evaluation of the sensitivity. The formed resist pattern was observed from above the pattern with use of the scanning electron microscope. The variation in the line width was measured at a total of 500 points. The 3 sigma value was obtained from the distribution of the measurement values, and defined as LWR performance (nm). The smaller the value of the LWR is, the smaller the roughness of the line is, which is better. The LWR performance was evaluated to be “good” in a case of being 3.0 nm or less, and “poor” in a case of exceeding 3.0 nm.

### [Storage Stability]

The positive radiation-sensitive composition for ArF immersion exposure was stored at 35° C. for 30 days, and then the optimum exposure amount for forming a 60 nm line-and-space pattern, that is, the sensitivity was measured again. When the change rate of the sensitivity (S₃₀) after storage for 30 days with respect to the sensitivity (S₀) before storage represented by the following formula was 0% or more and 1.0% or less, the storage stability was evaluated as “A” (extremely good), when the change rate was more than 1.0% and 2.0% or less, the storage stability was evaluated as “B” (good), and when the change rate was more than 2.0%, the storage stability was evaluated as “C” (poor).

Change rate of sensitivity (%)=(S₃₀−S₀)/S₀|×100

TABLE 5

| | Radiation- | | | |
|---|---|---|---|---|
| | sensitive | Sensitivity | LWR | Storage |
| | composition | (mJ/cm²) | (nm) | stability |
| Example 1 | J-1 | 25 | 2.6 | A |
| Example 2 | J-2 | 26 | 2.8 | A |
| Example 3 | J-3 | 26 | 2.7 | A |
| Example 4 | J-4 | 24 | 2.4 | A |
| Example 5 | J-5 | 27 | 2.4 | A |
| Example 6 | J-6 | 24 | 2.6 | A |
| Example 7 | J-7 | 24 | 2.6 | A |
| Example 8 | J-8 | 26 | 2.8 | A |
| Example 9 | J-9 | 23 | 2.6 | A |
| Example 10 | J-10 | 24 | 2.7 | A |
| Example 11 | J-11 | 25 | 2.6 | A |
| Example 12 | J-12 | 27 | 2.7 | A |
| Example 13 | J-13 | 28 | 2.9 | A |
| Example 14 | J-14 | 26 | 2.5 | A |
| Example 15 | J-15 | 29 | 2.7 | A |
| Example 16 | J-16 | 25 | 2.7 | A |
| Example 17 | J-17 | 24 | 2.7 | A |
| Example 18 | J-18 | 26 | 2.6 | A |
| Example 19 | J-19 | 28 | 2.8 | A |
| Example 20 | J-20 | 24 | 2.7 | A |
| Example 21 | J-21 | 25 | 2.7 | A |
| Example 22 | J-22 | 22 | 2.3 | A |
| Example 23 | J-23 | 23 | 2.4 | A |
| Example 24 | J-24 | 26 | 2.7 | A |
| Example 25 | J-25 | 25 | 2.6 | A |
| Example 26 | J-26 | 20 | 2.3 | A |
| Example 27 | J-27 | 23 | 2.5 | A |
| Example 28 | J-28 | 20 | 2.1 | A |
| Example 29 | J-29 | 22 | 2.4 | A |
| Example 30 | J-30 | 23 | 2.3 | A |
| Example 31 | J-31 | 19 | 2.2 | A |
| Example 32 | J-32 | 18 | 2.1 | A |
| Example 33 | J-33 | 25 | 2.3 | A |
| Example 34 | J-34 | 20 | 2.1 | A |
| Example 35 | J-35 | 24 | 2.5 | A |
| Example 36 | J-36 | 26 | 2.5 | A |
| Example 37 | J-37 | 27 | 2.7 | A |
| Example 38 | J-38 | 25 | 2.6 | A |
| Comparative | CJ-1 | 35 | 3.4 | B |
| Example 1 | | | | |
| Comparative | CJ-2 | 36 | 3.5 | B |
| Example 2 | | | | |
| Comparative | CJ-3 | 39 | 3.6 | B |
| Example 3 | | | | |

As is apparent from the results in Table 5, the radiation-sensitive compositions of Examples were good in sensitivity, LWR performance, MEEF performance, and storage stability when used for ArF immersion exposure, whereas the radiation-sensitive compositions of Comparative Examples were inferior in each characteristic to Examples. Therefore, when the radiation-sensitive composition of Examples is used for ArF immersion exposure, a resist pattern having good storage stability and excellent LWR performance with optimum sensitivity can be formed.

### [Preparation of Positive Radiation-Sensitive Composition for ArF-Dry Exposure]

### Example 39

100 parts by mass of (A-1) as a polymer [A], 6.0 parts by mass of (B-1) as an onium salt compound (1) [B], 3.0 parts by mass of (D-6) as an acid diffusion controlling agent [D], and 3,230 parts by mass of a mixed solvent of (E-1)/(E-2)/(E-3) as a solvent [E] were mixed, and the mixture was filtered through a membrane filter having a pore size of 0.2 μm to prepare a radiation-sensitive composition (J-39).

### Examples 40 to 52 and Comparative Examples 4 to 6

Radiation-sensitive compositions (J-40) to (J-52) and (CJ-4) to (CJ-6) were prepared in the same manner as in Example 39 except that the components of the types and contents shown in the following Table 6 were used.

TABLE 6

| | | | | [B] Onium salt | | [D] Acid diffusion | | | |
|---|---|---|---|---|---|---|---|---|---|
| | | [A] Polymer | | compound (1) | | controlling agent | | [E] Solvent | |
| | Radiation- | | Content | | Content | | Content | | Content |
| | sensitive | | (parts by | | (parts by | | (parts by | | (parts by |
| | composition | Type | mass) | Type | mass) | Type | mass) | Type | mass) |
| Example 39 | J-39 | A-1 | 100 | B-1 | 6.0 | D-6 | 3.0 | E-1/E-2/E-3 | 2240/960/30 |
| Example 40 | J-40 | A-6 | 100 | B-1 | 6.0 | D-6 | 3.0 | E-1/E-2/E-3 | 2240/960/30 |
| Example 41 | J-41 | A-7 | 100 | B-1 | 6.0 | D-6 | 3.0 | E-1/E-2/E-3 | 2240/960/30 |
| Example 42 | J-42 | A-8 | 100 | B-1 | 6.0 | D-6 | 3.0 | E-1/E-2/E-3 | 2240/960/30 |
| Example 43 | J-43 | A-1 | 100 | B-1 | 6.0 | D-1 | 3.0 | E-1/E-2/E-3 | 2240/960/30 |
| Example 44 | J-44 | A-1 | 100 | B-1 | 6.0 | D-5 | 3.0 | E-1/E-2/E-3 | 2240/960/30 |
| Example 45 | J-45 | A-1 | 100 | B-3 | 6.0 | D-6 | 3.0 | E-1/E-2/E-3 | 2240/960/30 |
| Example 46 | J-46 | A-1 | 100 | B-5 | 6.0 | D-6 | 3.0 | E-1/E-2/E-3 | 2240/960/30 |
| Example 47 | J-47 | A-1 | 100 | B-6 | 6.0 | D-6 | 3.0 | E-1/E-2/E-3 | 2240/960/30 |
| Example 48 | J-48 | A-1 | 100 | B-8 | 6.0 | D-6 | 3.0 | E-1/E-2/E-3 | 2240/960/30 |
| Example 49 | J-49 | A-1 | 100 | B-10 | 6.0 | D-6 | 3.0 | E-1/E-2/E-3 | 2240/960/30 |
| Example 50 | J-50 | A-1 | 100 | B-1/b-2 | 3.0/3.0 | D-6 | 3.0 | E-1/E-2/E-3 | 2240/960/30 |
| Example 51 | J-51 | A-1 | 100 | B-1/b-4 | 3.0/3.0 | D-6 | 3.0 | E-1/E-2/E-3 | 2240/960/30 |
| Example 52 | J-52 | A-1 | 100 | B-5/B-10 | 3.0/3.0 | D-6 | 3.0 | E-1/E-2/E-3 | 2240/960/30 |
| Comparative | CJ-4 | A-1 | 100 | b-9 | 6.0 | D-6 | 3.0 | E-1/E-2/E-3 | 2240/960/30 |
| Example 4 | | | | | | | | | |
| Comparative | CJ-5 | A-1 | 100 | b-10 | 6.0 | D-6 | 3.0 | E-1/E-2/E-3 | 2240/960/30 |
| Example 5 | | | | | | | | | |
| Comparative | CJ-6 | A-1 | 100 | b-11 | 6.0 | D-6 | 3.0 | E-1/E-2/E-3 | 2240/960/30 |
| Example 6 | | | | | | | | | |

### <Formation of Resist Pattern Using Positive Radiation-Sensitive Composition for ArF-Dry Exposure>

Onto the surface of an 8-inch silicon wafer, an underlayer antireflection film forming composition (“ARC29” manufactured by Brewer Science Incorporated) was applied with use of a spin coater (“CLEAN TRACK ACT8” manufactured by Tokyo Electron Limited). The wafer was then heated at 205° C. for 60 seconds to form an underlayer antireflection film having an average thickness of 77 nm. The positive radiation-sensitive composition for ArF-Dry exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 250 nm. Next, a resist pattern having a line-and-space of 90 nm in line width was formed on this resist film using an ArF excimer laser exposure apparatus (“S306C” manufactured by Nikon Corporation) at NA=0.75 under an optical condition of Annular (σ=0.8/0.6). After the exposure, PEB (post exposure baking) was performed at 100° C. for 60 seconds. Thereafter, the resist film was developed with an alkali with use of a 2.38% by mass aqueous TMAH solution as an alkaline developer, followed by washing with water and further drying to form a positive resist pattern (90 nm line-and-space resist pattern).

### <Evaluation>

The resist pattern formed using the positive radiation-sensitive composition for ArF-Dry exposure was evaluated on sensitivity, LWR performance, and storage stability in accordance with the following methods. The results are shown in the following Table 7. A scanning electron microscope (“S-9380” manufactured by Hitachi High-Tech Corporation) was used for measuring the length of the resist pattern.

### [Sensitivity]

An exposure dose at which a 90 nm line-and-space pattern was formed in the aforementioned resist pattern formation using each of the positive radiation-sensitive compositions for ArF-Dry exposure was defined as an optimum exposure dose, and this optimum exposure dose was defined as sensitivity (mJ/cm²). In a case of being 30 mJ/cm² or less, the sensitivity was evaluated as “good”, and in a case of being more than 30 mJ/cm², the sensitivity was evaluated as “poor”.

### [LWR Performance]

A 90 nm line-and-space resist pattern was formed by irradiation with the optimum exposure dose obtained in the evaluation of the sensitivity. The formed resist pattern was observed from above the pattern with use of the scanning electron microscope. The variation in the line width was measured at a total of 500 points. The 3 sigma value was obtained from the distribution of the measurement values, and defined as LWR performance (nm). The smaller the value of the LWR is, the smaller the roughness of the line is, which is better. The LWR performance was evaluated to be “good” in a case of being 4.0 nm or less, and “poor” in a case of exceeding 4.0 nm.

### [Storage Stability]

The positive radiation-sensitive composition for ArF-Dry exposure was stored at 35° C. for 30 days, and then the optimum exposure amount for forming a 90 nm line-and-space pattern, that is, the sensitivity was measured again. When the change rate of the sensitivity (S₃₀) after storage for 30 days with respect to the sensitivity (S₀) before storage represented by the following formula was 0% or more and 1.0% or less, the storage stability was evaluated as “A” (extremely good), when the change rate was more than 1.0% and 2.0% or less, the storage stability was evaluated as “B” (good), and when the change rate was more than 2.0%, the storage stability was evaluated as “C” (poor).

Change rate of sensitivity (%)=(S₃₀—S₀)/S₀|×100

TABLE 7

| | Radiation- | | | |
|---|---|---|---|---|
| | sensitive | Sensitivity | LWR | Storage |
| | composition | (mJ/cm²) | (nm) | stability |
| Example 39 | J-39 | 25 | 3.6 | A |
| Example 40 | J-40 | 24 | 3.6 | A |
| Example 41 | J-41 | 24 | 3.6 | A |
| Example 42 | J-42 | 26 | 3.8 | A |
| Example 43 | J-43 | 27 | 3.7 | A |
| Example 44 | J-44 | 29 | 3.7 | A |
| Example 45 | J-45 | 28 | 3.8 | A |
| Example 46 | J-46 | 25 | 3.7 | A |
| Example 47 | J-47 | 22 | 3.3 | A |
| Example 48 | J-48 | 26 | 3.7 | A |
| Example 49 | J-49 | 25 | 3.6 | A |
| Example 50 | J-50 | 20 | 3.1 | A |
| Example 51 | J-51 | 23 | 3.3 | A |
| Example 52 | J-52 | 24 | 3.5 | A |
| Comparative | CJ-4 | 36 | 4.4 | B |
| Example 4 | | | | |
| Comparative | CJ-5 | 37 | 4.5 | B |
| Example 5 | | | | |
| Comparative | CJ-6 | 39 | 4.6 | B |
| Example 6 | | | | |

As is apparent from the results in Table 7, the radiation-sensitive compositions of Examples were good in sensitivity, LWR performance, and storage stability when used for ArF-Dry exposure, whereas the radiation-sensitive compositions of Comparative Examples were inferior in each characteristic to Examples. Therefore, when the radiation-sensitive compositions of Examples are used for ArF-Dry exposure, a resist pattern having good storage stability and excellent LWR performance with optimum sensitivity can be formed.

### [Preparation of Positive Radiation-Sensitive Composition for Extreme Ultraviolet (EUV) Exposure]

### Example 53

100 parts by mass of (A-12) as a polymer [A], 20.0 parts by mass of (B-1) as an onium salt compound (1) [B], 10.0 parts by mass of (D-4) as an acid diffusion controlling agent [D], 3.0 parts by mass (solid content) of (F-5) as a high fluorine-content polymer [F], and 6,110 parts by mass of a mixed solvent of (E-1)/(E-4) as a solvent [E] were mixed, and the mixture was filtered through a membrane filter having a pore size of 0.2 μm to prepare a radiation-sensitive composition (J-53).

### Examples 54 to 63 and Comparative Examples 7 to 9

Radiation-sensitive compositions (J-54) to (J-63) and (CJ-7) to (CJ-9) were prepared in the same manner as in Example 53 except that the components of the types and contents shown in the following Table 8 were used.

TABLE 8

| | | | | | | | | [F] High | | | |
|---|---|---|---|---|---|---|---|---|---|---|---|
| | | | | [B] Onium salt | | [D] Acid diffusion | | fluorine-content | | | |
| | | [A] Polymer | | compound (1) | | controlling agent | | polymer | | [E] Solvent | |
| | Radiation- | | Content | | Content | | Content | | Content | | Content |
| | sensitive | | (parts by | | (parts by | | (parts by | | (parts by | | (parts by |
| | composition | Type | mass) | Type | mass) | Type | mass) | Type | mass) | Type | mass) |
| Example 53 | J-53 | A-12 | 100 | B-1 | 20.0 | D-4 | 10.0 | F-5 | 3.0 | E-1/E-4 | 4280/1830 |
| Example 54 | J-54 | A-12 | 100 | B-3 | 20.0 | D-4 | 10.0 | F-5 | 3.0 | E-1/E-4 | 4280/1830 |
| Example 55 | J-55 | A-12 | 100 | B-5 | 20.0 | D-4 | 10.0 | F-5 | 3.0 | E-1/E-4 | 4280/1830 |
| Example 56 | J-56 | A-12 | 100 | B-7 | 20.0 | D-4 | 10.0 | F-5 | 3.0 | E-1/E-4 | 4280/1830 |
| Example 57 | J-57 | A-12 | 100 | B-8 | 20.0 | D-4 | 10.0 | F-5 | 3.0 | E-1/E-4 | 4280/1830 |
| Example 58 | J-58 | A-12 | 100 | B-10 | 20.0 | D-4 | 10.0 | F-5 | 3.0 | E-1/E-4 | 4280/1830 |
| Example 59 | J-59 | A-12 | 100 | B-1/b-5 | 10.0/10.0 | D-4 | 10.0 | F-5 | 3.0 | E-1/E-4 | 4280/1830 |
| Example 60 | J-60 | A-12 | 100 | B-5/B-10 | 10.0/10.0 | D-4 | 10.0 | F-5 | 3.0 | E-1/E-4 | 4280/1830 |
| Example 61 | J-61 | A-13 | 100 | B-1 | 20.0 | D-4 | 10.0 | F-5 | 3.0 | E-1/E-4 | 4280/1830 |
| Example 62 | J-62 | A-14 | 100 | B-1 | 20.0 | D-4 | 10.0 | F-5 | 3.0 | E-1/E-4 | 4280/1830 |
| Example 63 | J-63 | A-15 | 100 | B-1 | 20.0 | D-4 | 10.0 | F-5 | 3.0 | E-1/E-4 | 4280/1830 |
| Comparative | CJ-7 | A-12 | 100 | b-9 | 20.0 | D-4 | 10.0 | F-5 | 3.0 | E-1/E-4 | 4280/1830 |
| Example 7 | | | | | | | | | | | |
| Comparative | CJ-8 | A-12 | 100 | b-10 | 20.0 | D-4 | 10.0 | F-5 | 3.0 | E-1/E-4 | 4280/1830 |
| Example 8 | | | | | | | | | | | |
| Comparative | CJ-9 | A-12 | 100 | b-11 | 20.0 | D-4 | 10.0 | F-5 | 3.0 | E-1/E-4 | 4280/1830 |
| Example 9 | | | | | | | | | | | |

### <Formation of Resist Pattern Using Positive Radiation-Sensitive Composition for EUV Exposure>

Onto the surface of a 12-inch silicon wafer, an underlayer antireflection film forming composition (“ARC66” manufactured by Brewer Science Incorporated) was applied with use of a spin coater (“CLEAN TRACKACT12” manufactured by Tokyo Electron Limited). The wafer was then heated at 205° C. for 60 seconds to form an underlayer antireflection film having an average thickness of 105 nm. The positive radiation-sensitive composition for EUV exposure prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR02. After exposing, PEB was performed at 120° C. for 60 seconds. Thereafter, the resist film was developed with an alkali with use of a 2.38% by mass aqueous TMAH solution as an alkaline developer, followed by washing with water and further drying to form a positive resist pattern (25 nm line-and-space pattern).

### <Evaluation>

The resist pattern formed using the positive radiation-sensitive composition for EUV exposure was evaluated on sensitivity, LWR performance, and storage stability in accordance with the following methods. The results are shown in the following Table 9. A scanning electron microscope (“CG-5000” manufactured by Hitachi High-Tech Corporation) was used for measuring the length of the resist pattern.

### [Sensitivity]

An exposure dose at which a 25 nm line-and-space pattern was formed in the aforementioned resist pattern formation using the positive radiation-sensitive composition for EUV exposure was defined as an optimum exposure dose, and this optimum exposure dose was defined as sensitivity (mJ/cm²). In a case of being 40 mJ/cm² or less, the sensitivity was evaluated as “good”, and in a case of being more than 40 mJ/cm², the sensitivity was evaluated as “poor”.

[LWR performance]

A resist pattern was formed by adjusting a mask size so as to form a 25 nm line-and-space pattern by irradiation with the optimum exposure dose obtained in the evaluation of the sensitivity. The formed resist pattern was observed from above the pattern with use of the scanning electron microscope. The variation in the line width was measured at a total of 500 points. The 3 sigma value was obtained from the distribution of the measurement values, and defined as LWR performance (nm). The smaller the value of the LWR is, the smaller the wobble of the line is, which is better. The LWR performance was evaluated to be “good” in a case of being 4.0 nm or less, and “poor” in a case of exceeding 4.0 nm.

### [Storage Stability]

The positive radiation-sensitive composition for EUV exposure was stored at 35° C. for 30 days, and then the optimum exposure amount for forming a 90 nm line-and-space pattern, that is, the sensitivity was measured again. When the change rate of the sensitivity (S₃₀) after storage for 30 days with respect to the sensitivity (S₀) before storage represented by the following formula was 0% or more and 1.0% or less, the storage stability was evaluated as “A” (extremely good), when the change rate was more than 1.0% and 2.0% or less, the storage stability was evaluated as “B” (good), and when the change rate was more than 2.0%, the storage stability was evaluated as “C” (poor).

$$Change rate of sensitivity ( \% ) = \vert ( {S}_{30} - {S}_{0} ) / {S}_{0} \vert \times 100$$

TABLE 9

| | Radiation- | | | |
|---|---|---|---|---|
| | sensitive | Sensitivity | LWR | Storage |
| | composition | (mJ/cm²) | (nm) | stability |
| Example 53 | J-53 | 31 | 3.8 | A |
| Example 54 | J-54 | 33 | 3.8 | A |
| Example 55 | J-55 | 30 | 3.7 | A |
| Example 56 | J-56 | 27 | 3.3 | A |
| Example 57 | J-57 | 31 | 3.7 | A |
| Example 58 | J-58 | 30 | 3.6 | A |
| Example 59 | J-59 | 28 | 3.3 | A |
| Example 60 | J-60 | 29 | 3.5 | A |
| Example 61 | J-61 | 30 | 3.6 | A |
| Example 62 | J-62 | 29 | 3.6 | A |
| Example 63 | J-63 | 29 | 3.6 | A |
| Comparative | CJ-7 | 43 | 4.4 | B |
| Example 7 | | | | |
| Comparative | CJ-8 | 44 | 4.5 | B |
| Example 8 | | | | |
| Comparative | CJ-9 | 48 | 4.7 | B |
| Example 9 | | | | |

As is apparent from the results in Table 9, the radiation-sensitive compositions of Examples were good in sensitivity, LWR performance, and storage stability when used for EUV exposure, whereas the radiation-sensitive compositions of Comparative Examples were inferior in each characteristic to Examples.

[Preparation of Negative Radiation-Sensitive Composition for ArF Exposure, and Formation and Evaluation of Resist Pattern Using this Composition]

### Example 64

[A] 100 parts by mass of (A-1) as a polymer, [B] 12.0 parts by mass of (B-1) as the onium salt compound (1), [D] 10.0 parts by mass of (D-7) as an acid diffusion controlling agent, [F] 2.0 parts by mass (solid content) of (F-3) as a high fluorine-content polymer, and [E] 3,230 parts by mass of a mixed solvent of (E-1)/(E-2)/(E-3) (blending amount: 2240/960/30 parts by mass) as a solvent were mixed, and the mixture was filtered through a membrane filter having a pore diameter of 0.2 μm to prepare a radiation-sensitive composition (J-64).

Onto the surface of a 12-inch silicon wafer, an underlayer antireflection film forming composition (“ARC66” manufactured by Brewer Science Incorporated) was applied with use of a spin coater (“CLEAN TRACK ACT12” manufactured by Tokyo Electron Limited). The wafer was then heated at 205° C. for 60 seconds to form an underlayer antireflection film having an average thickness of 100 nm. The negative radiation-sensitive composition for ArF exposure (J-64) prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 90 nm. Next, this resist film was exposed through a mask pattern having a hole of 50 nm and a pitch of 100 nm using an ArF excimer laser immersion exposure apparatus (“TWINSCAN XT-1900i” manufactured by ASML) with NA of 1.35 under an optical condition of Annular (σ=0.8/0.6). After the exposure, PEB (post exposure baking) was performed at 100° C. for 60 seconds. Thereafter, the resist film was developed with an organic solvent using n-butyl acetate as an organic solvent developer, and dried to form a negative resist pattern (contact hole pattern with hole of 50 nm and pitch of 100 nm).

The resist pattern using the negative radiation-sensitive composition for ArF exposure was evaluated on sensitivity in the same manner as in the evaluation of the resist pattern using the positive radiation-sensitive composition for ArF exposure. In addition, CDU performance and storage stability were evaluated in accordance with the following methods.

### [CDU Performance]

Contact holes with a 50 nm hole and a 100 nm pitch were formed by irradiation with an optimum exposure dose determined in the evaluation of sensitivity. The formed resist pattern was observed from above the pattern with use of the scanning electron microscope. The variation of the diameters of the contact holes was measured at 500 points in total. The 3 sigma value was determined from the distribution of the measurement values, and defined as CDU (nm). The smaller the value of the CDU is, the smaller the roughness of the contact holes is, which is better. When the value was less than 3.5 nm, the CDU performance was evaluated to be “good”, and when the value was 3.5 nm or more, the CDU performance was evaluated to be “poor”.

### [Storage Stability]

The negative radiation-sensitive composition for ArF exposure was stored at 35° C. for 30 days, and then the optimum exposure amount for forming contact holes having 50 nm hole and 100 nm pitch, that is, the sensitivity was measured again. When the change rate of the sensitivity (S₃₀) after storage for 30 days with respect to the sensitivity (S₀) before storage represented by the following formula was 0% or more and 1.0% or less, the storage stability was evaluated as “A” (extremely good), when the change rate was more than 1.0% and 2.0% or less, the storage stability was evaluated as “B” (good), and when the change rate was more than 2.0%, the storage stability was evaluated as “C” (poor).

Change rate of sensitivity (%)=(S₃₀−S₀)/S₀|×100

As a result, the radiation-sensitive composition of Example 64 had good sensitivity, CDU performance, and storage stability even when a negative resist pattern was formed by ArF exposure.

[Preparation of Negative Radiation-Sensitive Composition for EUV Exposure, and Formation and Evaluation of Resist Pattern Using this Composition]

### Example 65

[A] 100 parts by mass of (A-15) as a polymer, [B] 30.0 parts by mass of (B-7) as the onium salt compound (1), [D] 10.0 parts by mass of (D-4) as an acid diffusion controlling agent, [F] 5.0 parts by mass (solid content) of (F-5) as a high fluorine-content polymer, and [E] 6,110 parts by mass of a mixed solvent of (E-1)/(E-4) (blending amount: 4280/1830 parts by mass) as a solvent were mixed, and the mixture was filtered through a membrane filter having a pore diameter of 0.2 μm to prepare a radiation-sensitive composition (J-65).

Onto the surface of a 12-inch silicon wafer, an underlayer antireflection film forming composition (“ARC66” manufactured by Brewer Science Incorporated) was applied with use of a spin coater (“CLEAN TRACK ACT12” manufactured by Tokyo Electron Limited). The wafer was then heated at 205° C. for 60 seconds to form an underlayer antireflection film having an average thickness of 105 nm. The negative radiation-sensitive composition for EUV exposure (J-65) prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR15. After exposing, PEB was performed at 120° C. for 60 seconds. Thereafter, the resist film was developed with an organic solvent using n-butyl acetate as an organic solvent developer, and dried to form a negative resist pattern (contact hole pattern with hole of 20 nm and pitch of 40 nm).

The resist pattern formed using the negative radiation-sensitive composition for EUV exposure was evaluated in the same manner as the resist pattern formed using the negative radiation-sensitive composition for ArF exposure. As a result, the radiation-sensitive composition of Example 65 had good sensitivity, CDU performance, and storage stability even when a negative resist pattern was formed by EUV exposure.

According to the radiation-sensitive composition and the method for forming a resist pattern described above, a resist pattern having good storage stability of the composition, good sensitivity to exposure light, and excellent LWR performance and CDU performance can be formed. Therefore, the composition and method can be suitably used for a machining process and the like of a semiconductor device that is expected to be further miniaturized in the future.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising:
an onium salt compound represented by formula (1);
a polymer comprising a structural unit (I) which comprises an acid-dissociable group; and
a solvent, 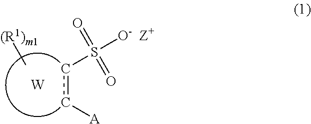
wherein,
W is a cyclic structure having 3 to 40 ring members formed together with the two carbon atoms,
a formula between the two carbon atoms below represents a single bond or a double bond, [symbol]
A is a group represented by formula (A-1), a group represented by formula (A-2), a group represented by formula (A-3), a group represented by formula (A-4), a group represented by formula (A-5), a group represented by formula (A-6), or a group represented by formula (A-7), 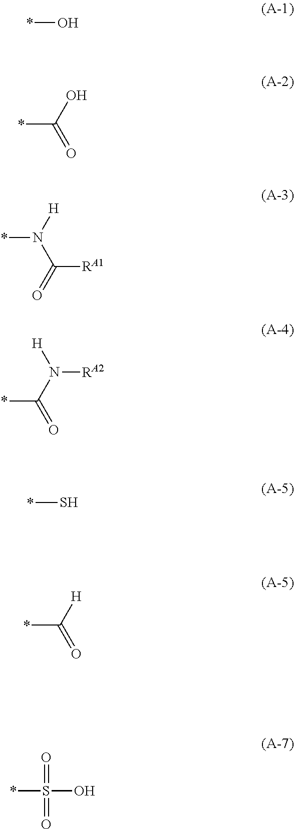
wherein, in the formulas (A-3) and (A-4), R^{A1} and R^{A2} are each independently a monovalent hydrocarbon group having 1 to 20 carbon atoms, and * is a bond with a carbon atom,
R¹ is a monovalent organic group having 1 to 20 carbon atoms, a cyano group, a nitro group, a carboxy group, a hydroxy group, an amino group, a halogen atom, or a thiol group, when there are a plurality of R¹s, the plurality of R¹s are same as or different from each other,
m₁ is an integer of 0 to 4, and
Z⁺ is a monovalent radiation-sensitive onium cation.

2. The radiation-sensitive composition according to claim 1, wherein W is an alicyclic hydrocarbon structure having 3 to 20 carbon atoms, an aromatic hydrocarbon structure having 6 to 20 carbon atoms, an aliphatic heterocyclic structure having 3 to 20 carbon atoms, or an aromatic heterocyclic structure having 5 to 20 carbon atoms.

3. The radiation-sensitive composition according to claim 1, wherein a content of the onium salt compound in the radiation-sensitive composition is 1 part by mass or more and 30 parts by mass or less with respect to 100 parts by mass of the polymer.

4. The radiation-sensitive composition according to claim 1, wherein
the structural unit (I) is represented by formula (3), 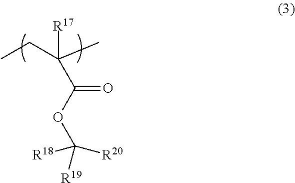
wherein,
R¹⁷ is a hydrogen atom, a fluorine atom, a methyl group, or a trifluoromethyl group,
R¹⁸ is a monovalent hydrocarbon group having 1 to 20 carbon atoms, and
R¹⁹ and R²⁰ are each independently a monovalent chain hydrocarbon group having 1 to 10 carbon atoms or a monovalent alicyclic hydrocarbon group having 3 to 20 carbon atoms, or R¹⁹ and R²⁰ taken together represent a divalent alicyclic group having 3 to 20 carbon atoms formed together with the carbon atom to which R¹⁹ and R²⁰ are bonded.

5. The radiation-sensitive composition according to claim 1, wherein
the structural unit (I) is represented by formula (3-1), 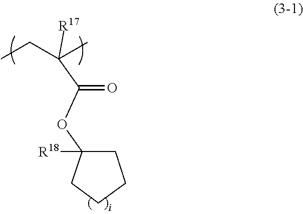
wherein,
R¹⁷ is a hydrogen atom, a fluorine atom, a methyl group, or a trifluoromethyl group,
R¹⁸ is an isopropyl group, a t-butyl group, or a phenyl group, and
i is an integer of 1 to 4.

6. The radiation-sensitive composition according to claim 1, wherein a content ratio of the structural unit (I) in the polymer with respect to all structural units constituting the polymer is 40 mol % or more and 80 mol % or less.

7. The radiation-sensitive composition according to claim 1, wherein the polymer further comprises a structural unit (II) comprising at least one selected from the group consisting of a lactone structure, a cyclic carbonate structure, and a sultone structure.

8. The radiation-sensitive composition according to claim 7, wherein a content ratio of the structural unit (II) in the polymer with respect to all structural units constituting the polymer is 20 mol % or more and 60 mol % or less.

9. The radiation-sensitive composition according to claim 1, further comprising an acid diffusion controlling agent.

10. A method for forming a pattern, comprising:
directly or indirectly applying the radiation-sensitive composition according to claim 1 to a substrate to form a resist film;
exposing the resist film to light; and
developing the exposed resist film with a developer.

11. The method for forming a pattern according to claim 10, wherein the resist film is exposed to light from an ArF excimer laser or extreme ultraviolet rays.

12. An onium salt compound represented by formula (1), 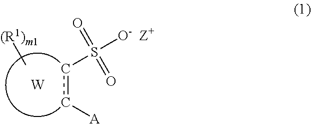
wherein,
W is a cyclic structure having 3 to 40 ring members formed together with the two carbon atoms,
a formula between the two carbon atoms below represents a single bond or a double bond, [symbol]
A is a group represented by formula (A-1), a group represented by formula (A-2), a group represented by formula (A-3), a group represented by formula (A-4), a group represented by formula (A-5), a group represented by formula (A-6), or a group represented by formula (A-7), 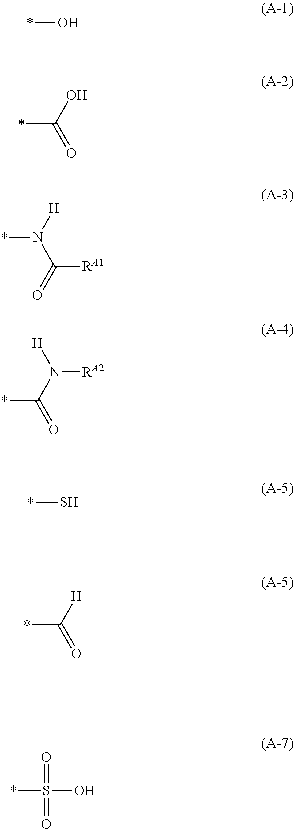
wherein, in the formulas (A-3) and (A-4), R^{A1} and R^{A2} are each independently a monovalent hydrocarbon group having 1 to 20 carbon atoms, and * is a bond with a carbon atom,
R¹ is a monovalent organic group having 1 to 20 carbon atoms, a cyano group, a nitro group, a carboxy group, a hydroxy group, an amino group, a halogen atom, or a thiol group, when there are a plurality of R¹s, the plurality of R¹s are same as or different from each other,
m₁ is an integer of 0 to 4, and
Z⁺ is a monovalent radiation-sensitive onium cation.
